(19) United States
(12) Patent Application Publication
(10) Pub. No.: US 2006/0002887 A1
(43) Pub. Date: Jan. 5, 2006

(54) Ionene oligomers and polymers

(75) Inventors: Richard J. Fitzpatrick, Marblehead, MA (US); Keith K. Shackett, Athol, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Genzyme Corporation, Cambridge, MA (US)

(21) Appl. No.: 11/131,944

(22) Filed: May 18, 2005

Related U.S. Application Data
(63) Continuation of application No. PCT/US03/36938, filed on Nov. 19, 2003.
(60) Provisional application No. 60/427,513, filed on Nov. 19, 2002.

(51) Int. Cl. A61K 31/765

(52) U.S. Cl. 424/78.3; 514/231.8; 514/316; 514/332

(57) ABSTRACT

Polymerized ionene compounds are known to be effective antimicrobial substances. It has recently been appreciated that the molecular weight can affect the safety and efficacy of ionene compounds. In particular, it has been found that low molecular weight ionene oligomers (less than 50 repeat units) are less toxic than larger polymers with identical compositions. The invention discloses a plurality of ionene oligomers.

### RELATED APPLICATION

This application is a continuation of International Application No. PCT/US03/36938, which designated the United States, was filed on Nov. 19, 2003, and was published in English, which claims the benefit of U.S. Provisional Application No. 60/427,513, filed on Nov. 19, 2002. The entire teachings of the International Application and U.S. Provisional Application are hereby incorporated herein by reference.

### BACKGROUND OF THE INVENTION

Infectious entities such as bacteria, fungi and protozoa, as well as viruses and organisms such as algae are capable of growing on a wide variety of living and non-living surfaces, including skin, teeth, mucosa, vascular tissue, medical implants, and medical devices. Invasive viral, parasitic and microbial infections of living organisms (e.g. bacterial, protozoal, fungal, etc.) can affect various organs of the body. Such infections are generally treated with well-characterized agents that may be safely tolerated by the host organism. However, the resistance of pathogens to various antiviral, antiparasitic and antimicrobial agents has increased at an alarming rate rendering many important therapeutics for the treatment of infections ineffective. Viruses, parasites and microorganisms employ one or more modes of resistance, often rendering them polyresistant. In particular, a great need still exists for effective anti-infective agents for wound management and infections of the skin, oral mucosa and gastrointestinal tract.

Individual microorganisms not attached to or growing on a surface are referred to as “planktonic”. These planktonic organisms are responsible for invasive and disseminated infections in the host, when it is a living organism. Such planktonic organisms are the targets of conventional antimicrobial therapy.

When planktonic microorganisms grow and disseminate on non-living surfaces, they may cause contamination and biofouling of that surface. In many cases a microorganism can grow and accumulate on a surface to the point of becoming almost impossible to remove. This accumulation takes place through the formation of biofilms. A biofilm occurs when one or more microorganisms attach to a surface and secrete a hydrated polymeric matrix that surrounds them. Microorganisms existing in a biofilm, termed sessile, grow in a protected environment that insulates them from attack from antimicrobial agents. These sessile communities can give rise to nonsessile planktonic organisms, which rapidly multiply and disperse over the surface. Once again, it is these planktonic organisms that are the targets of conventional antimicrobial treatments such as antibacterial and antifungal agents. However, these conventional treatments may fail to eradicate the sessile communities rooted in the biofilm. Biofilms are understood to be a frequently occurring reservoir for infectious agents and pose tremendous problems for the health-care industry. The biology of biofilms is described in more detail in “Bacterial biofilms: a common cause of persistent infection”, J. Costerson, P. Steward, E. Greenberg, Science 284: 1318-1322 (1999).

Microbial and pathogenic contamination and biofilms adversely affect the health care industry and other industries wherein microbial contamination poses a health risk to humans such as public water supplies, and food production facilities. Infections involving implanted medical devices, for example, generally involve biofilms, where a sessile community provides a reservoir for an invasive infection. Antibodies and host immune defenses are ineffective in killing the organisms contained in a biofilm even though these organisms have elicited the antibody and related immune response. Antibiotics typically treat the infection caused by the planktonic organisms, but may fail to kill those sessile organisms protected in the biofilm. Therefore, even if the contaminated medical device were removed from the host, any replacement device will be particularly susceptible to contamination from the residual microorganisms in the area from which the medical device was removed.

Since the difficulties associated with eliminating biofilm-based infections and contamination are well recognized, a number of technologies have developed to prevent or impair biofilm formation. These technologies include the development of various biocidal agents that are brought in contact with the contaminated or susceptible surface. However, any agent used to impair biofilm formation must be safe for use by humans and other non-target organisms. Biocides known to be effective at eliminating growth of unwanted microorganisms are generally toxic or otherwise harmful to humans, animals or other non-target organisms. Biocides known to be safe to non-target organisms, are generally less effective at preventing or eliminating microorganism growth, and require frequent application to the target surface.

Thus there is a need for antiparasitic, antiviral and antimicrobial agents that are non-toxic, long-lasting and effective at controlling contamination and infection by unwanted microbial organisms, with minimal development of resistant or polyresistant microorganisms.

### SUMMARY OF THE INVENTION

It has now been found that oligomeric polyionenes having a molecular weight of about 1-3 kilodaltons are antimicrobials, and, in many cases, are less toxic than the corresponding ionene polymer with a molecular weight of greater than 3 kilodaltons (Examples 21-23). Therefore, the present application discloses a plurality of small polymers (oligomers) and the use thereof as antivirals, antiparasitics and antimicrobial agents.

One embodiment of the present invention is a composition comprising a compound represented by Structural Formula (I): 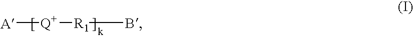
or a salt thereof, wherein:
-A′ is —R₁′ or —R₁-Q;
—B′ is -Q⁺-R₁′ or -Q;
each —R₁— is a linker and is independently selected;
—R₁′ is a substituted or unsubstituted hydrocarbyl group optionally interrupted with one or more heteroatoms; and each Q⁺ is independently represented by a structural formula selected from: 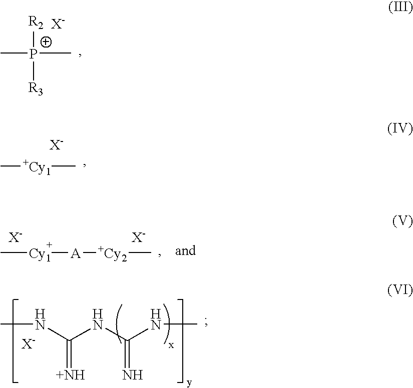
-Q is represented by a structural formula selected from: 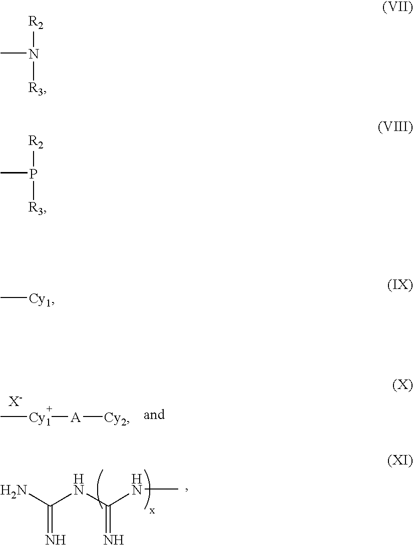
wherein tertiary phosphorus atoms of Structural Formula (VIII), tertiary nitrogen atoms of Structural Formulas (VII), (IX) and (X) and primary nitrogen atoms of Structural Formula (XI) are optionally alkylated or protonated; each Cy₁⁺ and Cy₂⁺ is independently a quaternary nitrogen-containing monocyclic heteroaromatic ring, a protonated tertiary nitrogen-containing non-aromatic heterocyclic ring or a quaternary nitrogen-containing non-aromatic ring;
each Cy₁ and Cy₂ is independently a nitrogen-containing non-aromatic heterocyclic ring or a nitrogen-containing heteroaromatic ring which is optionally alkylated or protonated;
A is a covalent bond, or a substituted or unsubstituted lower alkylene group;
R₂ and R₃ are independently —H or a substituted or unsubstituted aliphatic or aromatic group;
each X⁻, separately or taken together with other X⁻s, is an anion;
k is an integer from 1 to 25;
x is an integer from 0-4; and
y is an integer from 1-5.

In another embodiment, the invention is a polymer comprising repeat units represented by Structural Formula (XX): 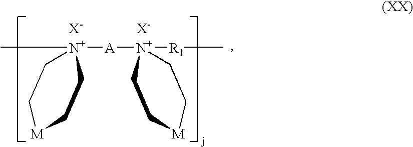
wherein:
each —R₁— is independently a linker;
A is a substituted or unsubstituted lower alkylene or arylene group;
M is (CH₂)ₜ, O, N, S, SO or SO₂;
each X⁻, separately or taken together with other X⁻s, is an anion;
j is a positive integer; and
t is 0 or 1.

The present invention also includes a polymer comprising repeat units represented by Structural Formula (XXI): 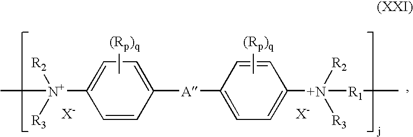
wherein:
   each —R₁— is independently a linker;
   R₂ and R₃ are independently —H or a substituted or unsubstituted aliphatic or aromatic group;
   Rₚ is independently —H, a halogen or a substituted or unsubstituted alkyl or alkoxy group;
   A″ is a covalent bond, or a substituted or unsubstituted lower alkylene or alkenylene group, preferably of 2 or more carbons; and
   each X⁻, separately or taken together with other X⁻s, is an anion; and
   j is a positive integer.

In another aspect, the present invention includes a pharmaceutical composition comprising a carrier or diluent and an ionene oligomer or polymer as described above. In pharmaceutical compositions of the present invention, Q can additionally be represented by Structural Formula (II): 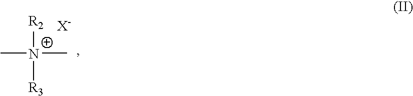
where R₂ and R₃ are as defined above.

In addition, the present invention is directed to a method of inhibiting colonization by a virus, parasite or microbe or treating a viral, parasitic or microbial infection in a mammal comprising the step of administering to the mammal an effective amount of one of the ionene oligomer or polymer compounds or compositions described herein. Other methods encompassed by the present invention include a method of inhibiting the colonization of a surface by a virus, parasite or microorganism or growth of a virus, parasite or microorganism on a surface comprising the step of contacting the surface with an effective amount of an ionene oligomer or polymer disclosed herein, a method of treating mucositis in a mammal comprising the step of administering to the mammal an effective amount of an ionene oligomer or polymer disclosed herein, and a method of preventing or inhibiting colonization or preventing or treating infection in a cystic fibrosis patient comprising the step of administering to the patient an effective amount of an ionene oligomer or polymer disclosed herein.

The invention has the advantage of providing polymeric compounds that are effective in treating a variety of conditions, but have decreased toxicity as compared to polymers with larger molecular weights. The present compounds are safe when administered to the gastrointestinal tract and the lungs.

### BRIEF DESCRIPTION OF THE DRAWINGS

FIG. 1 shows the combined histopathology scores for three ionene oligomers or oligomer/polymer mixtures in lung tissue obtained by the method of Example 23.

FIG. 2 shows the antimicrobial activity of two conventional antibiotics and 3 polyionenes in the diluted sputum of cystic fibrosis patients. The compound identified as 336-040-0003 is poly(trimethylene dipyridine-alt-octane) (TMDP-C₈). The compound identified as 456-069-0006 is poly(trimethylene dipyridine-alt-5-oxanonane). The compound identified as 461-170-0000 is a 3.5-mer of TMDP-C₈, where the terminal groups are each trimethylenedipyridine.

FIG. 3 shows that poly(trimethylene dipyridine-alt-5-oxanonane) (identified as 456-069-6) reduces the bacterial load in a chronic Pseudomonas aeruginosa infection model.

### DETAILED DESCRIPTION OF THE INVENTION

“Ionene oligomers” and “ionene polymers” (collectively ionenes or polyionenes), as used in the present invention, are cationic oligomers or polymers (including co-oligomers and copolymers) with quaternary nitrogen or phosphorus (e.g., having four carbons bonded to the nitrogen or phosphorus atom) or a protonated secondary or tertiary nitrogen or phosphorus located in the main polymeric chain or backbone of the polymer, providing a positive charge. Polyionenes can also be polyguanidines or copolymers thereof, where the cationic nitrogen atom is an imide nitrogen directly bonded to the polymer backbone. For the purposes of this application, polyionenes do not include polymers such as polyvinylamine, polyallylamine, polyallylamine and polyacrylamide. Additional ionene repeat units are disclosed in U.S. Ser. No. 60/262,586, filed Jan. 18, 2001, Ser. No. 10/051,765, filed Jan. 17, 2002, and Ser. No. 10/051,766, filed Jan. 17, 2002 (now U.S. Publication Nos. 2003/0021761 A1 and 2003/0031644 A1), the contents of which are incorporated herein by reference.

Polymers, as defined herein, contain 50 or more repeat units, preferably 50 to 500 repeat units. Oligomers, as defined herein, contain 1 to 49 repeat units, preferably 1 to 25 repeat units, even more preferably 1 to 15 repeat units and yet more preferably 1 to 10 (e.g., 1 to 4 or 2 to 4) or 4 to 10 repeat units. The molecular weight of ionene oligomers typically range from 500 to 5,000 Daltons, 1,000 to 5,000 Daltons, 500 to 3,000 Daltons, or 1,000 to 3,000 Daltons.

The present invention provides the use of the ionene polymers and oligomers disclosed herein in the treatment of a disease or condition disclosed herein. In addition, the invention also provides the use of the ionene polymers and oligomers disclosed herein in the manufacture of a medicament for the treatment of a disease or condition disclosed herein.

A “capping group” is a moiety at either or both termini of a polymer or oligomer that is present as a result of partial reaction of one of the starting materials. Specifically, ionene polymers and oligomers of the present invention can be prepared by reacting a divalent electrophile such as an α,ω-dihalogenated alkane or a corresponding diepoxide with a divalent nucleophile such as 4,4′-trimethylenedipiperidine or N,N,N′,N′-tetramethyl-1,3-propanediamine. When preparing a polyguanidine, the divalent nucleophile is an α,ω-diaminoalkane or an α,ω-aminoguanidine and the divalent electrophile typically is an α,ω-biscyanoguanidine. Polymerizing with one divalent electrophile and one divalent nucleophile results in a homopolymer. Polymerizing with two or more divalent electrophiles and/or divalent nucleophiles results in a copolymer. Such homopolymers and copolymers are encompassed within the present invention.

The “capping group” results from a partially reacted divalent electrophile or nucleophile or a monovalent electrophile or nucleophile. Partially reacted electrophiles are represented by the formulas —R₁′ and -Q⁺-R₁′. Partially reacted nucleophiles are represented by the formulas —R₁-Q and -Q. In one example, polymerizing 4,4′-trimethylenepyridine and 1,6-dibromohexane (or the corresponding epoxide) results in a polymer capped at either end with one of the following groups: 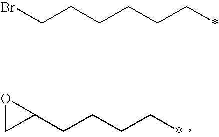
when the polymer terminates in either —R₁′ or -Q⁺-R₁′ (dependent upon which end of the polymer the capping group is located at); or 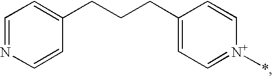
when the polymer terminates in either —R₁-Q or -Q (again dependent upon which end of the polymer the capping group is located at). In the first two capping groups shown, R₁′ is represented by —R₁—Y (e.g., R₁ of Structural Formula (I)), where Y is Br or an epoxide. Y is typically a leaving group, as defined below, or can be converted into another functional group.

The capping group can be reacted further. For example, capping groups that have a tertiary heteroatom (e.g., N, P) can be reacted with an alkylating agent (e.g., an alkyl halide or an alkyl group substituted with a different leaving group). Capping groups containing a tertiary heteroatom such as nitrogen or phosphorus (or a primary amine, in the case of guanidines) can also be reacted with an acid to protonate the heteroatom; suitable acids are listed below. Capping groups containing an oxiranyl group or a leaving group can be reacted with a nucleophile (e.g., water, hydride, alcohols, alkyl amines, aryl amines, alkyl phosphines, aryl phosphines). Leaving groups include halogens (e.g., bromine, chlorine, iodine), tosyl, triflyl, brosyl, p-nitrophenyl, 2,4-dinitrophenyl, and mesyl groups. Thus, capping groups represented by —R₁′ or -Q-R₁′ are often hydrocarbyl groups terminating (the terminal carbon or the two terminal carbons are substituted) in one of the aforementioned functional groups.

A linker group, as used herein, is a bond or a group that connects two cyclic structures. A linker group should be inert and should not adversely affect the properties of the molecule, e.g., decrease activity or increase toxicity. Linker groups are typically hydrocarbylene groups such as substituted or unsubstituted arylene or (straight chained lower) alkylene groups. Hydrocarbylene groups can be interrupted by one or more heteroatoms, such as in a polyethylene glycol group. Suitable substituents for a hydrocarbylene group are described in the section providing alkyl group substituents. A common substituent for a linker group is a hydroxyl group.

R₁ and R₁′ are preferably chosen such that R₁ is a substituted or unsubstituted alkylene group and R₁′ is a substituted alkyl group. R₁′ can be an unsubstituted alkyl group if it is reduced (e.g., with a hydride source) following polymer synthesis.

R₂ and R₃ are preferably each independently an alkyl group or a hydroxyalkyl group.

One preferred ionene oligomer has Q⁺ represented by Structural Formula (II) and Q represented by Structural Formula (VII). When Q⁺ is represented by Structural Formula (II), R₁ is preferably a substituted or unsubstituted phenylene, lower alkylene, polyalkylene glycol group, or —CH₂CHOH(CH₂)ₙCHOHCH₂—, where n is an integer ranging from 0 to 8, and R₂ and R₃ are independently an alkyl group or a hydroxyalkyl group. Even more preferably, R₁ is a substituted or unsubstituted straight chained lower alkylene group or polyalkylene glycol optionally substituted with one or more hydroxyl groups. When Q is represented by Structural Formula (VII), R₁′ is preferably a substituted or unsubstituted straight chained lower alkyl group or polyalkylene glycol, both of which can be substituted with one or more —OH, leaving groups or oxiranyl groups. In another preferred embodiment, R₁ and R₁′ of Structural Formulas (II) and (VII) are respectively an unsubstituted straight chained lower alkylene group and an alkyl group substituted with a leaving group.

Another preferred ionene oligomer has Q⁺ represented by Structural Formula (IV) and Q represented by Structural Formula (IX). An example of Cy₁⁺ is a piperidinium ring having a protonated tertiary nitrogen or a quaternary nitrogen additionally substituted with a substituted or unsubstituted lower alkyl group. An example of Cy₁ is a piperidine ring having a tertiary nitrogen substituted with a substituted or unsubstituted lower alkyl group. Preferably, the tertiary or quaternary nitrogen is substituted with a lower alkyl or hydroxy substituted lower alkyl group. An example of a “piperidinium” ring is represented in Structural Formula (XII): 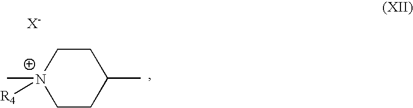
where R₄ is hydrogen or a substituted or unsubstituted lower alkyl group. An example of a “piperidine” capping group for an oligomer comprising piperidinium rings is represented by Structural Formula (XIII): 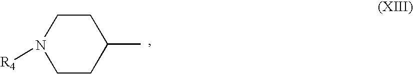
where R₄ is hydrogen or a substituted or unsubstituted lower alkyl group. Preferably, R₄ in both Structural Formulas (XII) and (XIII) is a lower alkyl or hydroxy-substituted lower alkyl group.

In another ionene oligomer, Q⁺ is represented by Structural Formula (V) and Q is represented by Structural Formula (X). In this oligomer, Cy₁⁺ and Cy₂+can each be piperidinium rings having a quaternary nitrogen substituted independently with a hydrogen or a substituted or unsubstituted lower alkyl group and A is as defined above. Analogously, Cy₁ and Cy₂ can be piperidine rings substituted independently with a hydrogen or a substituted or unsubstituted lower alkyl group and A is as defined above. More preferably, the nitrogen of a piperidine or piperidinium ring is substituted with a lower alkyl or hydroxy substituted lower alkyl group. An example of a “piperidinium” ionene of this type is represented in Structural Formula (XIV): 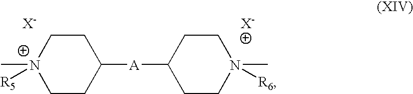
and a piperidine capping group is represented by Structural Formula (XV): 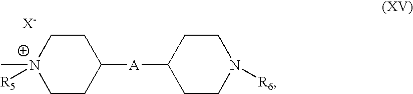
where A is as defined above, and R₅ and R₆ are each independently hydrogen or a substituted or unsubstituted lower alkyl group. Preferably, R₅ and R₆ are each independently an alkyl group or a hydroxyalkyl group, and A is an unsubstituted straight chained lower alkylene group. Even more preferably, A is an unsubstituted straight chained lower alkylene group.

In another embodiment, the ionene oligomer where Q⁺, represented by Structural Formula (V), is represented by the formula: 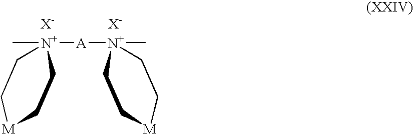
and where Q, represented by Structural Formula (X), is represented by the formula: 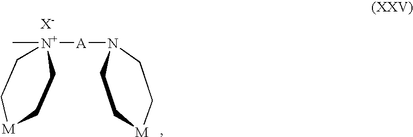
where A and M are as shown above and M is preferably O.

Another preferred ionene oligomer has Q⁺ represented by Structural Formula (V) and Q represented by Structural Formula (X), where Cy₁⁺ and Cy₂+are each pyridinium groups, Cy₂ is a pyridinyl group, and A is as defined above. In one example of a “pyridinium” ionene oligomer of this type, Q⁺ is characterized by Structural Formula (XVI): 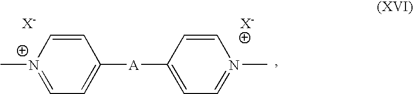
and Q is characterized by Structural Formula (XVII): 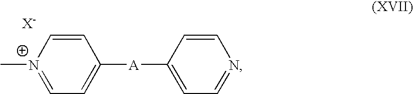
in which A and R₁ are as defined above. In a more preferred embodiment, A of Structural Formulas (XVI) and (XVII) is an unsubstituted straight chained lower alkylene group.

When Q⁺ is represented by Structural Formula (V), (XIV), (XVI) or (XXIV) and Q is represented by Structural Formula (X), (XV), (XVII) or (XXV), R₁ is preferably a substituted or unsubstituted straight chained lower alkylene or polyalkylene glycol group optionally substituted with one or more hydroxyl groups, more preferably an unsubstituted polyalkylene glycol or —CH₂CHOH(CH₂)ₙCHOHCH₂— where n is an integer ranging from 0 to 8; and R₁′ is preferably a substituted or unsubstituted lower alkyl group or polyalkylene glycol, which are optionally substituted with one or more —OH, leaving groups or oxiranyl groups. More preferably, R₁′ is a polyalkylene glycol group substituted with an oxiranyl group or an alkylene group substituted with an oxiranyl group and a hydroxyl group. In another preferred embodiment, R₁ and R₁′ of Structural Formulas (V), (XIV), (XVI) or (XXIV) and (X), (XV), (XVII) or (XXV), respectively, are an unsubstituted straight chained lower alkylene group and an alkyl group substituted with a leaving group, respectively.

A particularly preferred repeat unit with pyridinium rings is represented by Structural Formula (XVIII) or (XIX): 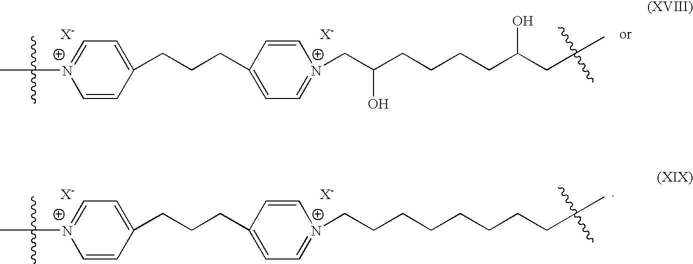
A preferred co-oligomer comprises or consists of repeat units represented by Structural Formulas (XVIII) and (XIX). Such copolymers can have alternating repeat units represented by Structural Formulas (XVIII) and (XIX). Alternatively, such copolymer can comprise about 45-55% each of repeat units represented by Structural Formulas (XVIII) and (XIX); about 30-40% of repeat units represented by Structural Formula (XVIII) and about 60-70% of repeat units represented by Structural Formula (XIX); about 60-70% of repeat units represented by Structural Formula (XVIII) and about 30-40% of repeat units represented by Structural Formula (XIX); about 23-27% of repeat units represented by Structural Formula (XVIII) and about 73-77% of repeat units represented by Structural Formula (XIX); or about 73-77% of repeat units represented by Structural Formula (XVIII) and about 23-27% of repeat units represented by Structural Formula (XIX).

In another preferred ionene oligomer, Q⁺ is represented by Structural Formula (III) or is represented by Structural Formula (II) and (III) and Q is represented by Structural Formula (VII) or (VIII). In such structures, R₂ and R₃ are preferably independently substituted or unsubstituted alkyl or aryl groups. When Q⁺ and Q are represented by Structural Formulas (III) and (VII) or (VIII), respectively, R₁ is preferably a substituted or unsubstituted straight chained lower alkylene or polyalkylene glycol group optionally substituted with one or more hydroxyl groups; and R₁′ is preferably a substituted or unsubstituted lower alkyl group or polyalkylene glycol, which are optionally substituted with one or more —OH, leaving groups or oxiranyl groups.

In yet another preferred ionene oligomer, Q⁺ is represented by Strucutral Formula (VI) and Q is represented by Structural Formula (XI). When Q⁺ and Q are represented by Structural Formulas (VI) and (XI), respectively, R₁ is preferably a substituted or unsubstituted straight chained lower alkylene or polyalkylene glycol group optionally substituted with one or more hydroxyl groups; and R₁′ is preferably a substituted or unsubstituted lower alkyl group or polyalkylene glycol, which are optionally substituted with one or more —OH, leaving groups or oxiranyl groups.

One type of ionene polymer is comprised of repeat units represented by Structural Formula (XX): 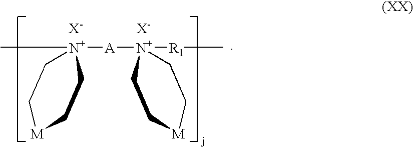
Preferably, j in Structural Formula (XX) is an integer from 50 to 500. R₁ is preferably a substituted or unsubstituted alkylene group and A is preferably a bond or a substituted or unsubstituted alkylene group. M is preferably (CH₂)ₜ, O, N or S, more preferably N or O and even more preferably O; t is 0 or 1. More preferably, j is an integer from 50 to 500, R₁ is a substituted or unsubstituted alkylene group, A is an unsubstituted alkylene group and M is O.

Polymers comprised of repeat units represented by Structural Formula (XX) can have capping groups represented by Structural Formulas (XXa)-(XXd): 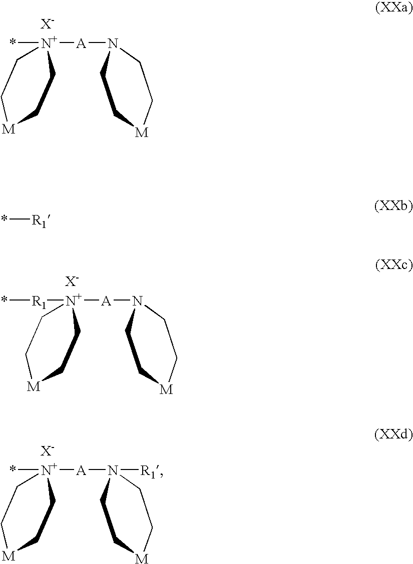
where A, M, X and R₁ are defined above and R₁′ is a substituted or unsubstituted hydrocarbyl group.

An example of an ionene polymer or oligomer is comprised of repeat units represented by Structural Formula (XXI): 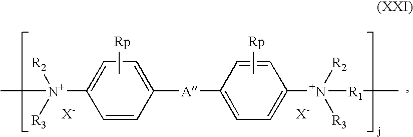
where R₁, R₂, R₃, Rₚ and X⁻ are as defined above and A″ is a substituted or unsubstituted alkylene or an alkenylene group. Polymers comprised of repeat units represented by Structural Formula (XXI) preferably have 50 to 500 repeat units. Oligomers comprised of repeat units represented by Structural Formula (XXI) preferably have from 1 to 49 repeat units, more preferably 2 to 25 repeat units and even more preferably 2 to 15 repeat units or 4 to 10 repeat units. Both oligomers and polymers can have capping groups represented by Structural Formula (XXII), (XXIII), (XXVI) or (XXVII): 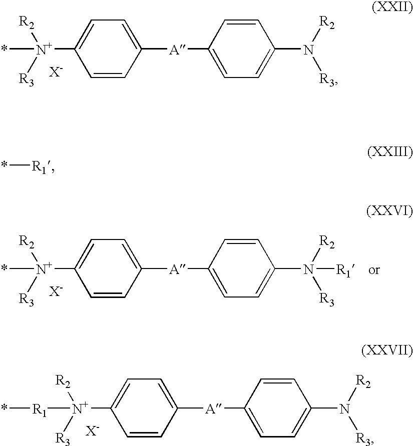
where R₁′ is a substituted or unsubstituted hydrocarbyl group and * represents where the capping group is attached to the polymer and A″, R₁, R₂, R₃ and X⁻ are as defined above. These polymers and oligomers are preferably characterized by one, two or three of the following features: 1) R₁ is a substituted or unsubstituted alkylene group, 2) A″ is a bond or a substituted or unsubstituted alkylene or alkenylene group (e.g, a methylene, ethylene, ethenylene, propylene, propenylene, butylene or butenylene group), and 3) R₂ and R₃ are each independently an alkyl group or a hydroxyalkyl group. Preferably, polymers and oligomers have Features 1 and 2, more preferably Features 1, 2 and 3.

Polymers and oligomers having repeat units represented by Structural Formula (XXI) can be prepared by reacting an α,ω-4-aminophenyl substituted alkane with an appropriate electrophilic compound (e.g., α,ω-dihaloalkanes, α,ω-diepoxyalkanes). Functionalization of capping groups and crosslinking of “aniline-like” polymers and oligomers can be carried out as described above for other polyionenes.

An “aliphatic group” is non-aromatic, consists solely of carbon and hydrogen and may optionally contain one or more units of unsaturation, e.g., double and/or triple bonds. An aliphatic group may be straight chained, branched, or cyclic and typically contains between about 1 and about 24 carbon atoms, more typically between about 1 and about 12 carbon atoms.

Aliphatic groups are preferably lower alkyl groups, lower alkylene or lower alkenylene groups, which include C1-24 (preferably C1-C12) straight chained or branched saturated hydrocarbons. An alkyl group is a saturated hydrocarbon in a molecule that is bonded to one other group in the molecule through a single covalent bond from one of its carbon atoms. Examples of lower alkyl groups include methyl, ethyl, n-propyl, iso-propyl, n-butyl, sec-butyl and tert-butyl. An alkoxy group is an alkyl group where an oxygen atom connects the alkyl group and one other group. An alkylene group is a saturated hydrocarbon in a molecule that is bonded to two other groups in the molecule through single covalent bonds from two of its carbon atoms. Examples of lower alkylene groups include methylene, ethylene, propylene, iso-propylene (—CH(CH₂)CH₂—), butylene, sec-butylene (—CH(CH₃)CH₂CH₂—), and tert-butylene (—C(CH₃)₂CH₂—). An alkenylene group is similar to an alkylene group, but contains one or more double bonds.

Aromatic groups include carbocyclic aromatic groups such as phenyl, 1-naphthyl, 2-naphthyl, 1-anthracyl and 2-anthacyl, and heterocyclic aromatic groups such as N-imidazolyl, 2-imidazolyl, 2-thienyl, 3-thienyl, 2-furanyl, 3-furanyl, 2-pyridyl, 3-pyridyl, 4-pyridyl, 2-pyrimidyl, 4-pyrimidyl, 2-pyranyl, 3-pyranyl, 3-pyrazolyl, 4-pyrazolyl, 5-pyrazolyl, 2-pyrazinyl, 2-thiazolyl, 4-thiazolyl, 5-thiazolyl, 2-oxazolyl, 4-oxazolyl and 5-oxazolyl.

Aromatic groups also include fused polycyclic aromatic ring systems in which a carbocyclic aromatic ring or heteroaryl ring is fused to one or more other heteroaryl rings. Examples include 2-benzothienyl, 3-benzothienyl, 2-benzofuranyl, 3-benzofuranyl, 2-indolyl, 3-indolyl, 2-quinolinyl, 3-quinolinyl, 2-benzothiazolyl, 2-benzooxazolyl, 2-benzimidazolyl, 2-quinolinyl, 3-quinolinyl, 1-isoquinolinyl, 3-quinolinyl, 1-isoindolyl and 3-isoindolyl. Phenyl is a preferred aromatic group.

“Arylene” is an aromatic ring(s) moiety in a molecule that is bonded to two other groups in the molecule through single covalent bonds from two of its ring atoms. Examples include phenylene —[—(C₆H₄₎—], thienylene [—(C₄H₂S)—] and furanylene [—(C₄H₂O)—].

A nitrogen-containing non-aromatic heterocyclic group is a cyclic group containing one or more nitrogen atoms in the ring, which can have one or more degrees of unsaturation provided that the group is not aromatic. Examples of nitrogen-containing non-aromatic heterocyclic groups include aziridine, azetidine, pyrrolidine, 2,3-1H-pyrrole, piperidine, morpholine, thiomorpholine, 1,2,3,4-tetrahydropyridine and 1,4-dihydropyridine.

A polyalkylene glycol is an alkylene group, which includes one or more ether linkages, where the chain includes a total of about 1 to about 12 carbon and oxygen atoms, and is optionally substituted with one or more hydroxyl groups. Preferably, the polyalkylene glycol is polyethylene glycol or polypropylene glycol.

A “hydrocarbyl group” is an alkylene or arylene group, i.e., —(CH₂)ₓ— or —(CH₂)ₓC₆H₄(CH₂)ₓ—, where x is a positive integer (e.g., from 1 to about 30), preferably between 6 and about 30, more preferably between 6 and about 15. The carbon chain of the hydrocarbyl group may be optionally interrupted with one or more ether (—O—), thioether (—S—), amine [—N(R^{a})—] or ammonium [—N⁺(R^{a}R^{b})—] linkages, or a combination thereof. R^{a} and R^{b} are independently —H, alkyl, substituted alkyl, phenyl, or substituted phenyl. R^{a} and R^{b} can be the same or different, but are typically the same. Examples of hydrocarbyl groups include butylene, pentylene, hexylene, heptylene, octylene, nonylene, decylene, dodecylene, 4-oxaoctylene, 5-oxanonylene, 4-azaoctylene, 4-thiaoctylene, 3,6-dioxaoctylene, 3,6-diazaoctylene, and 4,9-dioxadodecane.

Examples of suitable substituents on a hydrocarbyl, aliphatic, aromatic or benzyl group may include, for example, halogen (—Br, —Cl, —I and —F), —OR, —CN, —NO₂, —NR₂, —COOR, —CONR₂, —SOₖR (k is 0, 1 or 2), and —NH—C(═NH)—NH₂. An aliphatic group can also have ═O or ═S as a substituent. Each R is independently —H, an aliphatic group, a substituted aliphatic group, a benzyl group, a substituted benzyl group, an aromatic group or a substituted aromatic group, and preferably —H, a lower alkyl group, a benzylic group or a phenyl group. Substituent groups can be selected such that all substituents are either neutral or positively charged. A substituted benzylic group or aromatic group can also have an aliphatic or substituted aliphatic group as a substituent. A substituted aliphatic group can also have a benzyl, substituted benzyl, aromatic or substituted aromatic group as a substituent. A substituted hydrocarbyl, aliphatic, substituted aromatic or substituted benzyl group can have more than one substituent. A preferred substituent on an aliphatic group is —OH.

The anions represented by X⁻ in the polymer or oligomer can be the same or different. Each X⁻ in a repeat unit can separately be a monovalent anion, i.e., an anion having a negative charge of one. Alternatively, two or more X⁻s in the same repeat unit or in different repeat units, taken together, can represent an anion having a negative charge of two, three or more. A polymer or oligomer can comprise anions of different charges. Examples of suitable counteranions include sulfate, bisulfate, sulfite, bisulfite, phosphate, monohydrogenphosphate, dihydrogenphosphate, metaphosphate, pyrophosphate, chloride, bromide, iodide, acetate, proprionate, decanoate, caprylate, acrylate, formate, isobutyrate, caproate, heptanoate, propiolate, oxalate, malonate, succinate, fumarate, maleate, benzoate, sulfonate, phenylacetate, citrate, lactate, glycolate, tartrate, carbonate, bicarbonate and the like.

One anion can be exchanged for a second anion by various methods described in U.S. Application No. 60/397,868 and PCT Application No. PCT/US03/22514, the contents of which are incorporated herein by reference. In one such method, a proportion of the first anions of the ionene polymer can be exchanged for another anion by dissolving the polyionene in a solution containing the second anion or a mixture of the first and second anions. Another anion exchange method involves contacting the polyionene with an anionic exchange resin loaded with the desired second anion. Ion exchange processes involving an anionic exchange resin can be carried out in a throw-away mode, a regenerative mode, or in a continuous counter-current mode in simulated moving bed (SMB) equipment. In a further method, a proportion of the first anions of the polyionene can be exchanged for a second anion by electrodialysis. In electrodialysis, for example, a polyionene solution and a solution containing a salt having a desired second anion are passed through alternate channels of a stack of cation and/or anion exchange membranes. Conditions such as voltage, current density, flow rate of the solutions, and operation in co- or counter-current mode are controlled to produce a polyionene with the desired anion content. Polyionenes that have had their anions altered by any of the previously described methods can be purified by ultrafiltering the polyionene. Typically, ultrafiltration occurs simultaneously with or following anion exchange. For processes involving electrodialysis, ultrafiltration typically occurs prior to electrodialysis. Ultrafiltering a polyionene typically includes one or more cycles of diluting and concentrating the polyionene, whereby anions not bound to the polyionene and other contaminants are forced through a membrane and removed during concentration.

Also included in the present invention are physiologically acceptable salts of the oligomers having guanidine repeat units or repeat units comprising primary, secondary or tertiary phosphorus or nitrogen atoms. Salts can be formed by reacting the oligomer with a suitable acid. Examples include the corresponding acid of the counteranions listed above. Oligomers with guanidine repeat units can have up to one molecule of an acid such as hydrochloride or hydrobromide for every —NHC(═NH)NH— group in the repeat unit.

Ionene oligomers of the invention and pharmaceutical compositions thereof provide numerous advantages over conventional therapies for treatment of microbial infections. As used herein, “conventional antimicrobial” therapies include but are not limited to well known antibacterial agents, such as vancomycin, metronidazole, penicillin, oxacillin, streptomycin, rifamycin, amphotericin B, griseofulvin, penicillin, cephalothin, cefazolin, chloramphenicol, fluconazole, clindamycin, erythromycin, bacitracin, vancomycin, ciprofloxiacin, tertracycline, and fusidic acid, as well as antifungals, antiseptics and the like. Ionene polymers and oligomers of the invention provide a broader spectrum of treatment than presently available antibiotics. Ionene polymers and oligomers are less likely to elicit antibiotic resistance or polyresistance. When desirable, ionene polymers and oligomers of the invention may be designed such that they are not likely to be systemically absorbed by the body thus providing an attractive drug safety profile (e.g., sufficiently large size to exclude absorption by diffusion or pinocytosis or sufficient charge density to prevent diffusion across cell membranes).

Effective amounts of an ionene polymer or oligomer to be administered will be determined on an individual basis, and will be determined at least in part, by consideration of the individual's size, the severity of symptoms to be treated and the result sought. As used herein, an effective amount refers to an appropriate amount of active ingredient (ionene polymer or oligomer) to obtain therapeutic or prophylactic effect and can be determined by standard pharmaceutical procedures in cell cultures or experimental animals. Typical dosages range from between about 0.05 μg/kg body weight to about 500 mg/kg body weight, more typically between about 0.1 μg/kg body weight to about 100 mg /kg body weight and even more typically even more typically between about 0.5 μg/kg body weight and about 10 mg/kg body weight.

The polymer or oligomer can be administered alone or in a pharmaceutical composition comprising the polymer or oligomer, a pharmaceutically acceptable carrier, and optionally, one or more additional drugs. The polymers or oligomers can be administered, for example, topically, ophthalmically, vaginally, orally, buccally, intranasally, by aerosol, rectally, by injection (e.g., intramuscular, intraperitoneal, subcutaneous) or by pulmonary means. The form in which the polymer or oligomer is administered, for example, powder, tablet, capsule, solution, or emulsion, depends in part on the route by which it is administered. Suitable carriers and diluents will be immediately apparent to persons skilled in the art. These carrier and diluent materials, either inorganic or organic in nature, include, for example, gelatin, albumin, lactose, starch, magnesium stearate preservatives (stabilizers), melting agents, emulsifying agents, salts and buffers. For topical administration, examples of pharmaceutically acceptable carriers include, for example, commercially available inert gels, or liquids supplemented with albumin, methyl cellulose or a collagen matrix. Typical of such formulations are ointments, creams and gels. The effective amount can be administered in a series of doses separated by appropriate time intervals such as minutes or hours.

Pathogenic infections which can be treated or prevented or inhibited (e.g., by preventing or inhibiting colonization) by administering an effective amount of an ionene polymer or oligomer or a pharmaceutical composition thereof to a mammal infected with a microbe include, but are not limited to, bacterial infections, such as infection by species of Streptococcus, Salmonella, Campylobacter, Helicobacter, Burkholderia, Actinomyces, Eschericha, Mycobacteria, Pasturella, Francisella, Clostridium, Staphylococcus, Shigella, Pseudomonas, Listeria, Bacillus, Eikenella, Actinobacillus, Bacteriodes, Capnocytophaga, Wolinella, Bacteriodes, Mycoplasma, Treponema, Peptostreptococcus, Bacteriodes, Fusobacteria, Selenomonas, Bacteriodes, and Enterobacter. Specific species include Eschericha coli, Clostridium difficile, Eikenella corrodens, Actinobacillus actinomycetemcomitans, Bacteriodes gingivalis, Wolinella recta, Bacteriodes intermedius, Peptostreptococcus micros, Bacteriodes forsythus, Selenomonas sputigena, Bacteriodes fragilis, and Enterobacter cloacae. Other pathogenic infections include viral infections, protozoal infections, mycoplasma infections, fungal infections, and parasitic infections. The growth of these pathogens on a surface or the colonization of a surface can be inhibited by methods described in detail below.

In one preferred embodiment, polymers and oligomers are administered to the oral cavity for treatment of infections and ulcers of the mouth. In another preferred embodiment, polymers and oligomers of the invention are administered orally for treatment of infections in the gastrointestinal tract of a mammal. In yet another preferred embodiment, polymers and oligomers of the invention are administered topically for treatment of ocular infections or for treatment of infections on the skin of a mammal. One example of treatment of infections on the skin of a mammal is a wound management regimen that includes a polymer or oligomer of the invention alone or in combination with a tissue sealant or other wound repair product as is known in the art.

One type of condition that can be advantageously treated with the disclosed polymers and oligomers is mucositis. Mucositis is defined herein as inflammation and/or ulceration of a mucous membrane, which can be caused by infection, abrasion, radiation injury, chemical injury, antineoplastics, antibodies or other tissue injury. The disclosed method can be used to treat mucositis in the stomach, intestines, and the like; however, it is particularly effective when used to treat oral mucositis. Oral mucositis is characterized by inflammation of a mucous membrane of the oral cavity or lips and is typically accompanied by redness, swelling, and/or ulcerations of the mouth. Included in this description is oral mucositis that is a side-effect of anti-cancer therapies such as chemotherapy and radiotherapy, and oral mucositis that is a side effect of bone marrow transplantation or stem cell transplant or ablation. Treatment with an ionene oligomer can be particularly beneficial for patients undergoing treatment for tumors of the head and neck, such as radiation patients. Mucositis also includes mucositis that develops spontaneously in a healthy patient not receiving anti-cancer therapy, as in the case of a canker sore or mouth ulcer.

Treatment of mucositis includes both prophylactic and therapeutic uses of the ionene polymers and oligomers. Desired prophylactic effects include prevention of and inhibition of mucositis, reduction in severity of mucositis, reduction in size of mucositis lesions compared with, for example, what is normally experienced by a mammal undergoing cancer therapy, and reduction in likelihood of developing mucositis.

Desired therapeutic effects include amelioration of the discomfort associated with the oral mucositis, and/or increased rate of healing of mucositis lesions compared with, for example, what is normally experienced by a mammal undergoing cancer therapy. Thus, the invention provides, in one aspect, a method of treating mucositis or oral mucositis comprising administering an effective amount of an ionene polymer or oligomer.

For prophylactic treatment of mucositis resulting from chemotherapy, treatment with an ionene polymer or oligomer is initiated before the onset of the chemotherapy, during chemotherapy, after chemotherapy is complete but before symptoms appear or any combination of the above. For prophylactic treatment of mucositis resulting from radiation therapy, treatment with the ionene polymer or oligomer is initiated before the onset of radiation therapy, during radiation exposure, after radiation exposure has been terminated (preferably no sooner than about one hour, more preferably five hours after termination) but before symptoms appear or treatment can occur in any combination of the above time periods. For therapeutic treatment of mucositis resulting from radiation therapy or chemotherapy, the ionene polymer or oligomer is administered after symptoms of mucositis (e.g., mouth ulcers) have appeared.

The polymer or oligomer for mucositis therapy can be administered alone or in a pharmaceutical composition comprising the polymer or oligomer, a pharmaceutically acceptable carrier, and optionally, one or more additional drugs, e.g., antibiotics or antimicrobials. Examples include streptomycin, rifamycin, amphotericin B, griseofulvin, penicillin, cephalothin, cefazolin, chloramphenicol, fluconazole, clindamycin, erythromycin, bacitracin, vancomycin, ciprofloxiacin, tetracycline, and fusidic acid.

The polymers or oligomers for mucositis therapy can be administered, for example, topically, orally, buccally, intranasally, by aerosol, rectally or vaginally, depending upon the site of the inflamed tissue or ulcer. The form in which the polymer or oligomer is administered, for example, powder, tablet, capsule, solution, or emulsion, depends in part on the route by which it is administered. For oral mucositis, the polymer or oligomer is preferably administered orally as a gargle, an ointment, a swab, a gel, or the like.

Suitable carriers and diluents for an ionene polymer or oligomer used to treat mucositis will be immediately apparent to persons skilled in the art. These carrier and diluent materials, either organic or inorganic in nature, include, for example, gelatin, lactose, starch, magnesium stearate, preservatives (stabilizers), sugars, emulsifying agents, salts and buffers. When applied directly to the lesion, examples of pharmaceutically acceptable carriers include, for example, commercially available inert gels, or liquids supplemented with albumin, methyl cellulose, or a collagen matrix.

An effective amount of an ionene polymer or oligomer to treat mucositis to be administered will be determined on an individual basis, and will be determined at least in part, by consideration of the individual's size, the severity of symptoms to be treated and the result sought. As used herein, an effective amount refers to an appropriate amount of ionene polymer or oligomer, which results in a desired therapeutic or prophylactic effect with respect to mucositis, as defined above. Typical dosages for applied and/or ingested ionene polymers or oligomers range from between about 0.05 μg/kg body weight to about 500 mg/kg body weight, more typically between about 0.1 μg/kg body weight to about 100 mg/kg body weight and even more typically between about 0.5 μg/kg body weight and about 10 mg/kg body weight.

The method of treating mucositis is preferably used with human patients, but can also be used with other mammals, such as companion animals (e.g., dogs, cats, and the like), farm animals (horses, cattle, goats, and the like) and laboratory animals (hamsters, mice, rats, and the like).

In another preferred embodiment, polymers of the invention are administered by inhalation for treatment or prevention or inhibition of pulmonary infections. Pulmonary infections suitable for treatment with polymers disclosed herein include pneumonia, bronchopneumonia and bronchitis, which are caused by pathogens including Streptococcus pneumoniae, Streptococcus pyogenes, Staphylococcus aureus, Legionella spp. (L. pneumophila, L. micdadei), Chlamydia pneumoniae, Chlamydia psittaci, Hemophilus influenzae, Enterobacteriaceae, Pseudomonas aeruginosa, Escherichia coli, Fusobacterium nucleatum, Bacteroides melaminogenicus, Bacteroides fragilis, Mycobacterium tuberculosis, Bacillus anthracis, Yersinia pestis, Francisella tularensis, Coxiella burnetti, Enterobacter spp., Proteus spp., Klebsiella spp., Brucella spp., Mycoplasma spp., Blastomyces spp., Leptospira spp., Histoplasma spp., Coccidioides spp., Cryptococcus spp., Candida spp., Pneumocystis carinii, Entamoeba histolytica, the influenza A and B viruses, the measles virus, cytomegalovirus and adenovirus.

Particular pulmonary infections or colonizations for which the disclosed polymers and oligomers are effective include those which accompany cystic fibrosis. Patients suffering from cystic fibrosis (CF) produce excessive quantities of sweat and mucus. The mucus secreted is very thick and blocks passageways in the lungs and sinuses, causing them to be susceptible to colonization and/or infection by unwanted agents. Respiratory tract or pulmonary infections, which lead to respiratory inflammation and respiratory failure, are a primary cause of morbidity and mortality in CF patients. As there are currently no treatments to cure the root cause of CF (a defective protein, cystic fibrosis transmembrane conductance regulator), treatment of a patient suffering from CF with an ionene polymer or oligomer represents a means of addressing the complications associated with CF. Present therapies for CF-associated infections are often inadequate, as pathogens develop resistance to various therapies.

Pulmonary colonizations and infections typically become more prevalent and chronic as a patient suffering from CF ages (respiratory cultures from more than 80% of adults suffering from CF were positive for colonization by a pathogen). Therefore, the present method includes administering a polymer or oligomer of the present invention before colonization or before an infection is acquired to prevent or inhibit onset of an infection. The method also includes treating a CF patient who is suffering from an active infection.

Colonizations and infections associated with CF are typically caused by a large variety of pathogens including Gram negative bacteria, Gram positive bacteria, fungi and viruses capable of infecting respiratory tract tissues. Bacteria and fungi associated with CF include, but are not limited to, Pseudomonas, Staphylococcus, Haemophilus, Burkholderia, Aspergillus, Candida, Mycobacteria, Mycoplasma, Stenotrophomonas, Escherichia, Achromobacter, Ralstonia, Acinetobacter, Streptococcus, Flavobacterium, Alicaligenes, CDC group VB3 and Klebsiella. Specific microbial species causing the colonization or infection include Pseudomonas aeruginosa, Staphylococcus aureus, Haemophilus influenzae, Burkholderia cepacia, Aspergillus fumigatus, Candida albicans, Mycoplasma pneumoniae, Stenotrophomonas maltophilia, Escherichia coli, Ralstonia mannitolilytica, Ralstonia pickettii, Streptococus pneumoniae, Flavobacterium indologenes, Burkholderia gladioli, Acinetobacter baumannii, Acinetobacter hameolyticus, Achromobacter xylosoxidans and Klebsiella pneumoniae. Viruses associated with CF include influenza virus (e.g., influenza virus A, influenza virus B, influenza virus C), respiratory syncytical virus and Rhinovirus. Pseudomonas aeruginosa is preferably treated or inhibited by the present method.

The polymer can be administered alone or in a pharmaceutical composition comprising the polymer, a pharmaceutically acceptable carrier, and optionally, one or more additional drugs, e.g., antibiotics or antimicrobials. Examples of co-therapies for infections or complications due to CF include tobramycin and other aminoglycosides, ciprofloacin and other fluoroquinolones, rifabutin, ethambutol, clarithromycin, clofazimine, aztreonam, cephalothin, cefazolin, nafcillin, ticarcilin, clavulanate, gentamicin, amikacin, ceftazidime, piperacillin, imipenem, cefepime, chloramphenicol, colistin, dicloxacillin, cefaclor, amoxicillin, azithromycin, trmethoprim/sulfa, cefpodoxime, tetracyclines, amiloride and meropenem. These antibiotics can be administered orally, by injection or by pulmonary means. The term “pulmonary” as used herein refers to any part, tissue or organ whose primary function is gas exchange with the external environment, i.e., O₂/CO₂ exchange, within a patient. “Pulmonary” typically refers to the tissues of the respiratory tract. Thus, the phrase “pulmonary administration” refers to administering the formulations described herein to any part, tissue or organ whose primary function is gas exchange with the external environment (e.g., mouth, nose, pharynx, oropharynx, laryngopharynx, larynx, trachea, carina, bronchi, bronchioles, alveoli). For purposes of the present invention, “pulmonary” is also meant to include a tissue or cavity that is contingent to the respiratory tract, in particular, the sinuses.

The polymer or oligomer can also be administered with an anti-inflammatory drug or steroid such as ibuprofen, prednisone (corticosteroid) or pentoxifylline. Another suitable co-therapy is administering domase alfa (DNase), nacystelyn, gelsolin and hypertonic saline, which reduce mucus buildup, or administering a decongestant or bronchodilator (e.g, a beta adrenergic receptor agonist, an anticholinergic drug, theophylline).

The polymers and oligomers of the present invention can also be administered in CF therapy following a physical therapy that aids mucus drainage. Such treatments include chest physiotherapy (manual or mechanical). Manual techniques include autogenic drainage and percussive techniques. Devices for mechanical therapy include positive expiratory pressure treatment, the “Flutter” (a device that produces oscillations during exhalation), and an inflatable vest driven by a pulsed-air delivery system.

Polymers and oligomers of the present invention can be administering to a patient suffering from CF in a manner as described above, but are preferably administered by pulmonary means (e.g., aerosol), intranasally or orally.

Conventional means to deliver the active agent by pulmonary means to a patient include administration of an aerosol formulation containing the active agent from, for example, a manual pump spray, nebulizer or pressurized metered-dose inhaler.

Delivery of aerosolized therapeutics, particularly aerosolized antibiotics, is known in the art (see, for example U.S. Pat. No. 5,767,068 to VanDevanter et al., U.S. Pat. No. 5,508,269 to Smith et al., and WO 98/43650 by Montgomery, the entire teachings of which are incorporated herein by reference). Polymer and oligomer compositions of the invention to be delivered as aerosols for treatment of pulmonary infection are formulated such that an effective dose may be aerosolized (e.g., using a jet or ultrasonic nebulizer) to a particle size optimal for treatment of pulmonary infections. Examples of a suitable particle size for delivery into the endobronchial space is generally about 1 to 5 microns.

A drug delivery device for delivering aerosols comprises a suitable aerosol canister with a metering valve containing a pharmaceutical aerosol formulation as described and an actuator housing adapted to hold the canister and allow for drug delivery. The canister in the drug delivery device has a head space representing greater than about 15% of the total volume of the canister. Often, the polymer intended for pulmonary administration is dissolved, suspended or emulsified in a mixture of a solvent, surfactant and propellant. The mixture is maintained under pressure in a canister that has been sealed with a metering valve.

When administering the drug, the patient must actuate the drug delivery device. The actuation releases a fraction of the formulation from within the canister to the external environment. A force, created by vaporized propellant, expels the drug into the air and away from the device. The patient then inhales the aerosolized drug. The metering valve controls the amount of the formulation released, which, in turn, effectively controls the amount of drug available for inhalation by the patient.

Particles can also be administered by pulmonary means. To ensure that the drug particles have the proper size and shape, the particles may be analyzed using known techniques for determining particle morphology. For example, the particles can be visually inspected under a microscope and/or passed through a mesh screen. Preferred techniques for visualization of particles include scanning electron microscopy (SEM) and transmission electron microscopy (TEM). Particle size analysis may take place using laser diffraction methods. Commercially available systems for carrying out particle size analysis by laser diffraction are available from Clausthal-Zellerfeld, Germany (HELOS H11006).

Particles for pulmonary administration are typically substantially nonacicular particles. The particles will preferably have an average particle size in the range of about 0.5 micrometer to about 10 micrometer, more preferably in the range of about 1 micrometer to about 7.5 micrometer, and most preferably in the range of about 1 micrometer to about 5 micrometer. Preferably, greater than about 85%, more preferably greater than about 95%, and most preferably greater than about 98% of the population of particles in the formulation will fall within the desired particle size range, e.g., about 0.5 micrometer to about 10 micrometer, about 1 micrometer to about 7.5 micrometer, and soon.

Preferred drug delivery devices for particles are metered-dose inhalers. Metered-dose inhalers are described in Remington: The Science and Practice of Pharmacy, Twentieth Edition (Easton, Pa.: Mack Publishing Co., 2000) and in Ansel et al., Pharmaceutical Dosage Forms and Drug Delivery Systems, Sixth Edition (Malvern, Pa.: Lea & Febiger, 1995). The components of the drug delivery device, e.g., canister, housing, metering valve, etc., are commercially available. For example many components are available from 3M Corporation, St. Paul, Minn. Typically, although not necessarily, the amount of pharmaceutical formulation (including polymer, solvents and other expicipients) that is released per actuation of the drug delivery device is about 5 micrograms to about 100,000 micrograms of formulation.

The polymers and oligomers are formulated in a manner appropriate for the route of administration. Typical formulations for pharmaceutical compositions are described above.

A polymer or oligomer administered in the treatment or prevention of infection in CF patients generally has a weight between 500 and 5000 Daltons, 500 and 3000 Daltons, or 1000 and 3000 Daltons.

The ionene polymers and oligomers of the invention are also particularly useful for inhibiting the colonization and the growth and dissemination, of viruses, parasites and microorganisms, particularly on surfaces wherein such growth is undesirable. The term “inhibiting the growth of microorganisms” means that the growth, dissemination, accumulation, and/or the attachment, e.g. to a susceptible surface, of one or more viruses or microorganisms is impaired, retarded, eliminated or prevented. In a preferred embodiment, the polyionene compositions of the invention are used in methods for inhibiting the growth of an organism on susceptible surfaces in health-related environments. The term “health-related environment” as used herein includes all those environments where activities are carried out directly or indirectly, that are implicated in the restoration or maintenance of human health. A health-related environment can be a medical environment, where activities are carried out to restore human health. An operating room, a doctor's office, a hospital room, and a factory making medical equipment are all examples of health-related environments. Other health-related environments can include industrial or residential sites where activities pertaining to human health are carried out such as activities including food processing, water purification, recreational water maintenance, and sanitation.

The term “susceptible surface” as used herein refers to any surface whether in an industrial or medical setting, that provides an interface between an object and the fluid. A surface, as understood herein further provides a plane whose mechanical structure, without further treatment, is compatible with the adherence of microorganisms. Microbial growth and/or biofilm formation with health implications can involve those surfaces in all health-related environments. Such surfaces include, but are not limited to, scalpels, needles, scissors and other devices used in invasive surgical, therapeutic or diagnostic procedures; implantable medical devices, including artificial blood vessels, catheters and other devices for the removal or delivery of fluids to patients, artificial hearts, artificial kidneys, orthopedic pins, plates and implants; catheters and other tubes (including urological and biliary tubes, endotracheal tubes, peripherally insertable central venous catheters, dialysis catheters, long term tunneled central venous catheters, peripheral venous catheters, pulmonary catheters, Swan-Ganz catheters, urinary catheters, peritoneal catheters), urinary devices (including long term urinary devices, tissue bonding urinary devices, artificial urinary sphincters, urinary dilators), shunts (including ventricular or arterio-venous shunts); prostheses (including breast implants, penile prostheses, vascular grafting prostheses, heart valves, artificial joints, artificial larynxes, otological implants), vascular catheter ports, wound drain tubes, hydrocephalus shunts, pacemakers and implantable defibrillators, and the like.

Other surfaces include the inner and outer surfaces of pieces of medical equipment, medical gear worn or carried by personnel in the health care settings and protective clothing for biohazard or biological warfare applications. Such surfaces can include counter tops and fixtures in areas used for medical procedures or for preparing medical apparatus, tubes and canisters used in respiratory treatments, including the administration of oxygen, solubilized drugs in nebulizers, and anesthetic agents. Additional surfaces include those surfaces intended as biological barriers to infectious organisms such as gloves, aprons and faceshields.

Surfaces in contact with liquids are particularly prone to microbial growth or colonization and/or biofilm formation. As an example, those reservoirs and tubes used for delivering humidified oxygen to patients can bear biofilms inhabited by infectious agents. Dental unit waterlines similarly can bear biofilms on their surfaces, providing a reservoir for continuing contamination of the system of flowing and aerosolized water used in dentistry.

Other surfaces related to health include the inner and outer surfaces of equipment used in water purification, water storage and water delivery, and those articles involved in food processing equipment for home use, materials for infant care and toilet bowls.

In accordance with the invention, a method for preventing, inhibiting or eliminating the growth, dissemination, presence and/or accumulation of microorganisms on a susceptible surface (including but not limited to the formation of biofilms) comprises the step of contacting such surface with an polyionene agent, or composition thereof of the invention, with an amount sufficient to prevent, inhibit or eliminate such growth, dissemination, presence and/or accumulation, i.e., with an effective amount.

As used herein “contacting” refers to any means for providing the compounds of the invention to a surface to be protected from, microbial growth and/or biofilm formation. Contacting can include spraying, wetting, immersing, dipping, painting, bonding, coating, adhering or otherwise providing a surface with a compound or composition in accordance with the invention. A “coating” refers to any temporary, semipermanent, or permanent layer, covering a surface. A coating can be a gas, vapor, liquid, paste, semisolid or solid. In addition a coating can be applied as a liquid and solidify into a hard coating. Examples of coatings include polishes, surface cleaners, caulks, adhesives, finishes, paints, waxes, polymerizable compositions (including phenolic resins, silicone polymers, chlorinated rubbers, coal tar and epoxy combinations, epoxy resins, polyamide resins vinyl resins, elastomers, acrylate polymers, fluoropolymers, polyesters and polyurethane, and latex). Silicone resins, silicone polymers (e.g. RTV polymers) and silicone heat cured rubbers are suitable coatings for use in the invention and described in the art. Coatings can be ablative or dissolvable, so that the dissolution rate of the matrix controls the rate at which the compositions of the invention are delivered to the surface. Coatings can also be non-ablative, and rely on diffusion principals to deliver a composition of the invention to the target surface. Non-ablative coatings can be porous or non-porous. A coating containing an antimicrobial agent of the invention freely dispersed in a polymer binder is referred to as a “monolithic” coating. Elasticity can be engineered into coatings to accommodate pliability, e.g. swelling or shrinkage of the surface to be coated.

Other means for contacting include a sustained or controlled release system that provides constant or prolonged release of an agent of the invention from a susceptible surface. This can be accomplished through the use of diffusional systems, including reservoir devices in which a core of an agent of the invention is surrounded by a porous membrane or layer, and also matrix devices in which the compound is distributed throughout an inert matrix. Materials which may be used to form reservoirs or matrices include silicones, acrylates, methacrylates, vinyl compounds such as polyvinyl chloride, olefins such as polyethylene or polypropylene, fluoropolymers such as polytetrafluorethylene or polypropylene, fluoropolymers such as polytetrafluorethylene, and polyesters such as terephthalates. Alternatively, the compositions of the invention may be mixed with a resin, e.g., polyvinyl chloride and then molded into a formed article, which integrally incorporates the compound to form a structure having a porous matrix which allows diffusion of the compound or a functional portion thereof into the surrounding environment. Microencapsulation techniques can also be used to maintain a sustained focal release of a compound of the invention.

Other means for providing the polyionene agents of the invention to a susceptible surface will be apparent to those of skill in the art.

The compounds and compositions of the invention are also useful for preventing microbial growth and/or biofilms in industries outside of health-related industries, such as industrial systems wherein the presence of an aqueous environment leads to biofilm formation. Examples of such systems include metal working fluids, cooling waters (e.g. intake cooling water, effluent cooling water, recirculating cooling water), and other recirculating water systems such as those used in papermaking or textile manufacture. Marine industries are also plagued by unwanted biofilms such as those that form on boat hulls and other marine structures.

Another embodiment of the present invention is an article comprising a polymer or oligomer of the present invention in an amount sufficient to prevent, inhibit or eliminate the growth or dissemination of a microorganism or the formation of a biofilm, i.e., an “effective amount.” The polymer or oligomer can be in the article or on the surface of the article. Preferably, the article is coated with a composition comprising an effective amount of a polymer or oligomer of the present invention. Articles that are advantageously coated with a polymer or oligomer of the present invention are those in which inhibition of the growth of microorganisms and/or biofilms is desirable, e.g., medical devices, medical furniture and devices exposed to aqueous environments. Examples of such articles are described above.

Ionene polymers and oligomers of the present invention can be prepared, for example, by a reacting a divalent electrophile such as an α,ω-dihalogenated alkane or a corresponding diepoxide with a divalent nucleophile such as 4,4′-trimethylenedipiperidine or N,N,N′,N′-tetramethyl-1,3-propanediamine. When preparing a polyguanidine, the divalent nucleophile is an α,ω-diaminoalkane or an α,ω-aminoguanidine and the divalent electrophile typically is an α,ω-biscyanoguanidine. Polymerizing with one divalent electrophile and one divalent nucleophile results in a homopolymer. Polymerizing with two or more divalent electrophiles and/or divalent nucleophiles results in a copolymer. Such homopolymers and copolymers and the corresponding oligomers are encompassed within the present invention.

The size of polyionenes produced by the above method can be selected or controlled in two ways. In the first method, the reaction is allowed to proceed to completion, likely resulting in polyionenes of various molecular weights. A polyionene having a desired molecular weight or being within a desired range of molecular weights can be selected by several purification methods. Purification methods include ultrafiltration, gel permetation chromatography and HPLC. In one example, a mixture of polyionene products are filtered through a membrane having a 3 kDa molecular weight cutoff membrane, where the filtrate is kept and subsequently filtered through a 1 kDa molecular weight cutoff membrane where the retentate is the product having polyionenes with molecular weights ranging from 1 kDa to 3 kDa. The steps of this process can optionally be reversed.

The polyionenes prepared in U.S. Patent Application Nos. 60/262,586, filed Jan. 18, 2001, Ser. No. 10/051,765, filed Jan. 17, 2002 and Ser. No. 10/051,766, filed Jan. 17, 2002, the contents of which are incorporated herein by reference, can be processed by the method described above to result in an ionene oligomer of the desired molecular weight.

In the second method, the ratio of a diamine-containing monomer (e.g., a nucleophilic monomer) to a monomer having two leaving groups (e.g. an electrophilic monomer) is chosen such that a compound having a degree of polymerization within a desired range is obtained (e.g., to produce an oligomer having a molecular weight of about 1 kDa to about 3 kDa). Ionene polymers and oligomers of the invention can be cross-linked with primary, secondary or other polyfunctional amines using means known in the art. Ionene polymers and oligomers can be cross-linked by polymerizing in the presence of a multivalent nucleophile (i.e., a compound with three or more nucleophilic groups such as a triamine or tetraamine) or a multivalent electrophile (i.e., a compound with three or more nucleophilic groups such as a trihalide or tetrahalide).

Additional examples of repeat units (-Q⁺-R₁—) include those represented by the following structural formulas:

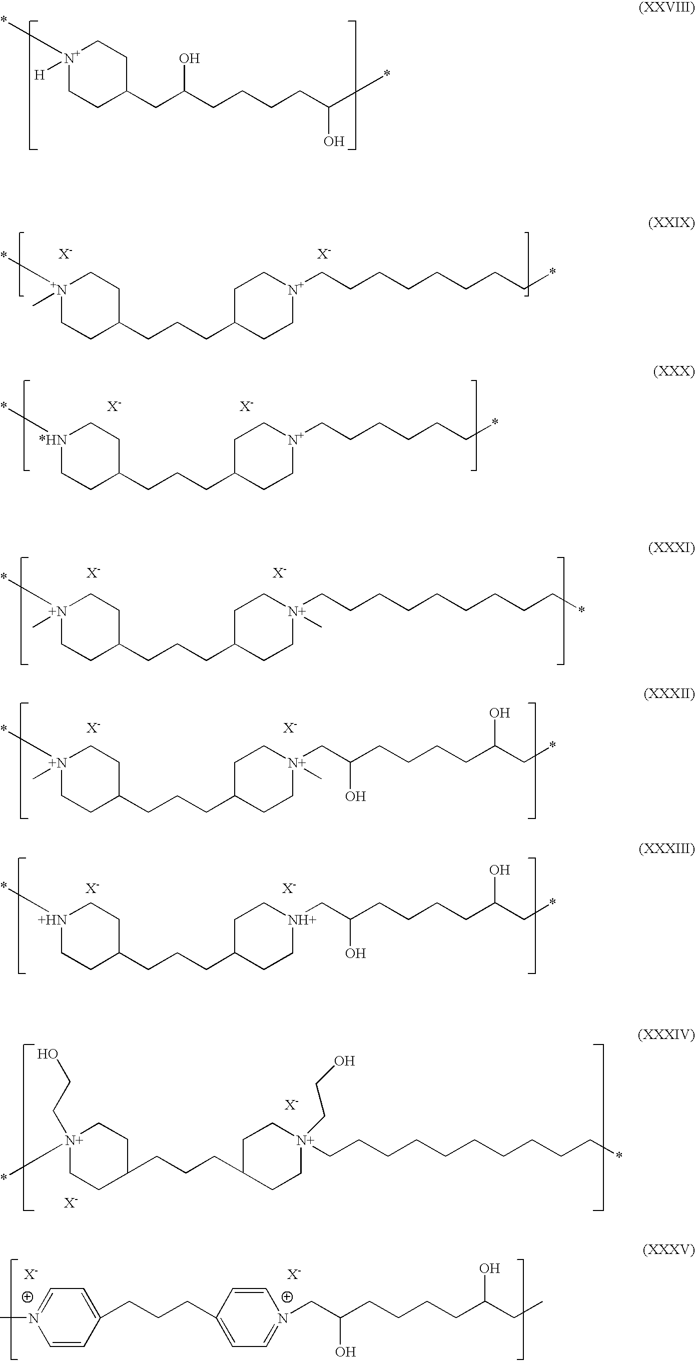

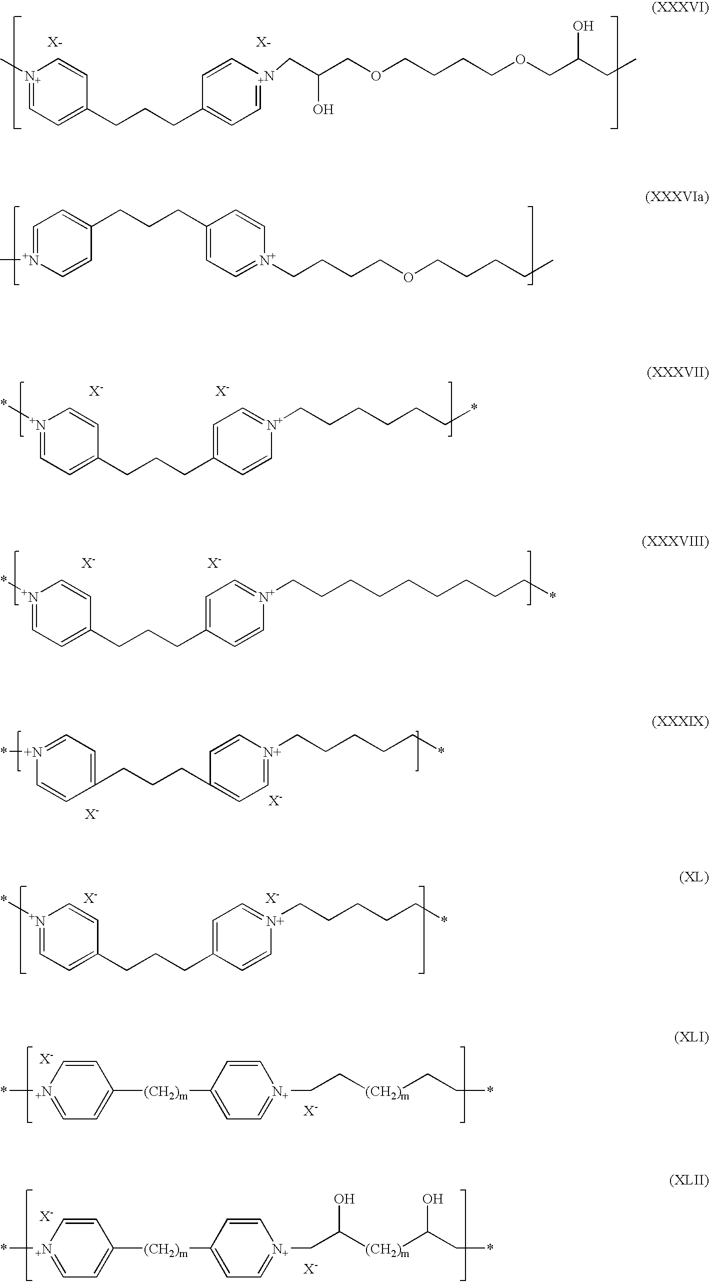

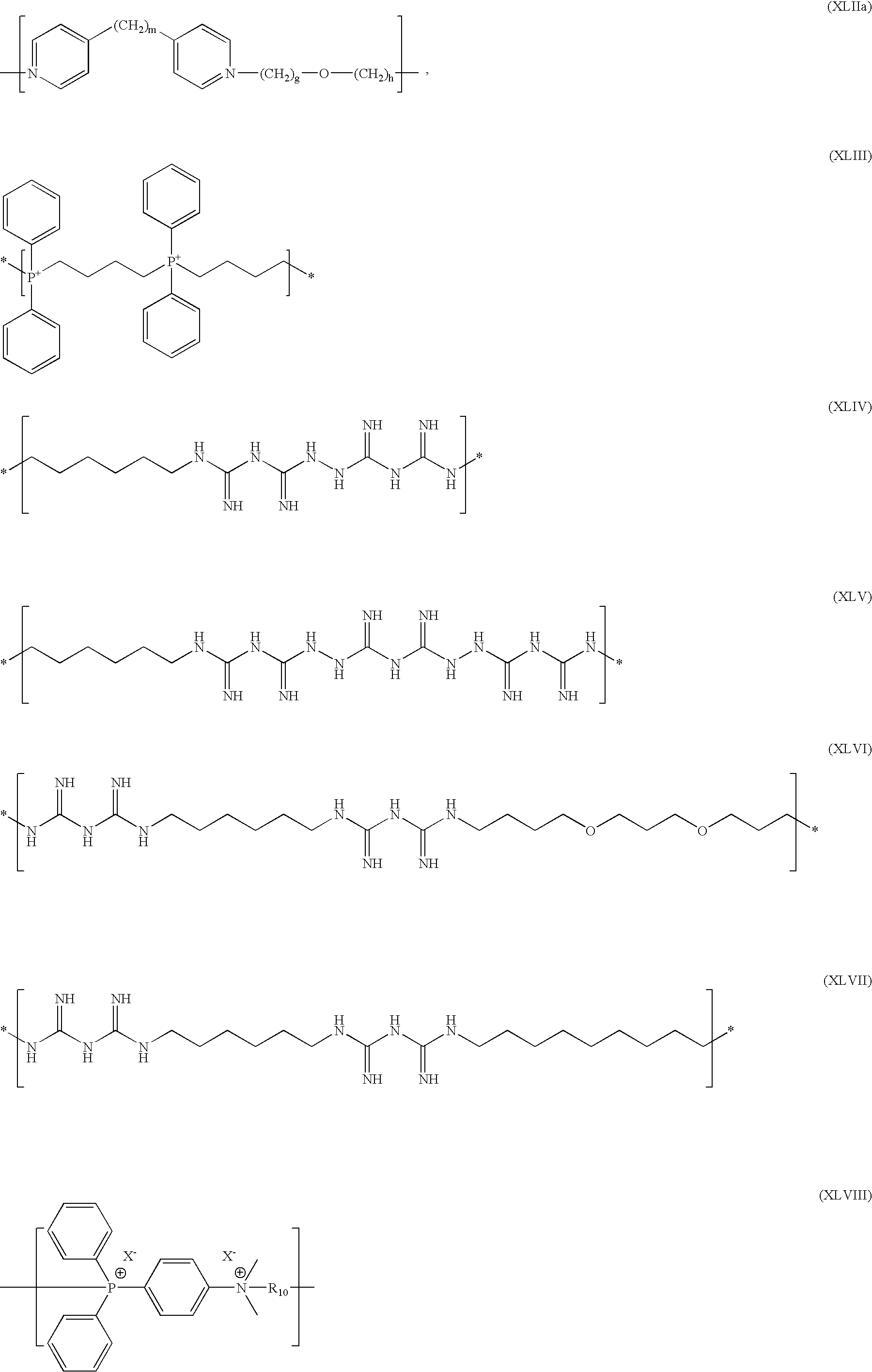

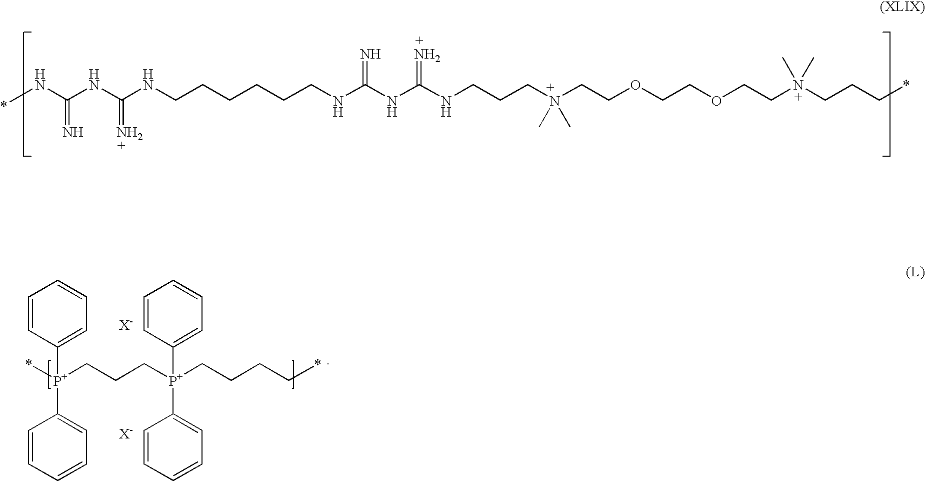

Other specific examples of repeat units of polyionenes that can be used in the disclosed method are represented by Structural Formulas (XLI) and (XLII) above, where m is 1 and n is 0; m is 1 and n is 1; m is 1 and n is 2; m is 1 and n is 4; m is 1 and n is 5; m is 1 and n is 6; m is 1 and n is 8; m is 2 and n is 0; m is 2 and n is 1; m is 2 and n is 2; m is 2 and n is 4; m is 2 and n is 5; m is 2 and n is 6; m is 2 and n is 8; m is 3 and n is 0; m is 3 and n is 1; m is 3 and n is 2; m is 3 and n is 4; m is 3 and n is 5; m is 3 and n is 6; m is 3 and n is 8; m is 4 and n is 0; m is 4 and n is 1; m is 4 and n is 2; m is 4 and n is 4; m is 4 and n is 5; m is 4 and n is 6; m is 4 and n is 8; m is 5 and n is 0; m is 5 and n is 1; m is 5 and n is 2; m is 5 and n is 4; m is 5 and n is 5; m is 5 and n is 6; and m is 5 and n is 8.

Specific examples of repeat units of polyionenes represented by Structural Formula (XLIIa) above include those where m is 1 and g and h are each 1; m is 1 and g and h are each 2; m is 1 and g and h are each 4; m is 1 and g and h are each 5; m is 1 and g and h are each 6; m is 2 and g and h are each 1; m is 2 and g and h are each 2; m is 2 and g and hare each 4; m is 2 and g and h are each 5; m is 2 and g and hare each 6; m is 3 and g and h are each 1; m is 3 and g and h are each 2; m is 3 and g and h are each 4; m is 3 and g and h are each 5; m is 3 and g and h are each 6; m is 4 and g and h are each 1; m is 4 and g and h are each 2; m is 4 and g and h are each 4; m is 4 and g and h are each 5; m is 4 and g and h are each 6; m is 5 and g and h are each 1; m is 5 and g and h are each 2; m is 5 and g and h are each 4; m is 5 and g and h are each 5; m is 5 and g and h are each 6; and m is 5 and g and h are each 8.

### EXEMPLIFICATION

### Example 1

### Preparation of Poly(trimethylene dipyridine-alt-2,7-dihydroxyoctane)

4,4′-trimethylenedipyridine (100 g) was transferred to the lower portion of a 1 L reaction flask. Neat liquid diepoxyoctane (71.72 g) was added to the reaction flask. The reactor contents were stirred under nitrogen at room temperature for ten minutes or until most of the 4,4′-trimethylenedipyridine has dissolved. Stirring neat acetic acid (121 g) was added dropwise through the constant addition funnel over a 24 hour period at room temperature under nitrogen. The reaction became dark blue and very viscous. The reaction mixture was stirred at room temperature for an additional 4 days. The reaction mixture was dissolved in deionized water (1 L). The pH was adjusted to about 0.8. The solution was heated to 50° C. and stirred at that temperature for 48 hours. The reaction mixture was allowed to cool, diluted with deionized water to 16 L, and then neutralized with NaHCO₃. The mixture was purified through a tangential flow ultrafiltration device (1K MWCO), reducing the volume by 8 L. 8 L of deionized water and 200 g of NaCl were added to the retentate. The mixture was again purified through a tangential flow ultrafiltration device (1K MWCO), reducing the volume by 8 L. Once again, 8 L of deionized water was added to the retentate. The steps of purifying the mixture through the tangential flow ultrafiltration device and re-diluting the retentate with 8 L of deionized water were repeated until the filtrate had a conductivity less than 0.1 mS/cm. The retentate was further purified by tangential flow filtration with a 3K MWCO membrane. This time the filtrate was collected, in order to obtain the desired “1-3K fraction” (theoretically 1-3 kilodalton molecular weight oligomers). The filtrate product was vacuum dried at 45° C. on a speed-vac.

### Example 2

### Preparation of Poly(trimethylene dipyridine-alt-2,7-dihydroxyoctane)

In a 1 L, 3-neck round bottom flask under N₂ with a temperature probe and 60 mL pressure equalizing funnel, 20.539 g (0.104 mol) of 4,4′-trimethylenedipyridine and 13.6 mL (0.093 mol) of 1,2,7,8-diepoxyoctane was added. The reaction was stirred for 1 hour under N₂. The dipyridine was not completely dissolved. Acetic acid (75 mL) was added over 0.5 hours through the pressure equalizing funnel. With addition of the acetic acid, the reaction temperature began to rise (reaching 42° C.), so the reaction was put in an ice bath to maintain the temperature below 30° C.

After 24 hours of stirring under N₂, an aliquot was removed for reverse phase HPLC. The HPLC indicated that the reaction had proceeded, therefore the flask was put in an ice bath and ion exchange was performed by adding 300 mL deionized H₂O and 300 mL of 37% HCl (3.65 mol, 2.8 times the CH₃COOH). The reaction was then heated to 50° C. After approximately 40 hours, the reaction was cooled and diluted to 2 L with deionized H₂O. NaHCO₃ (305 g, 3.63 mol) was added to raise the pH to 4.02.

The reaction volume was diluted from 2 L to 4.5 L and circulated in the tank (392-294-R, conductivity=68 mS/cm) of a Millipore Reverse Osmosis (RO) apparatus. Under a pressure of 150 psi, 2.25 L of permeate was collected (conductivity=73.6 mS/cm, V=2.25 L; conductivity=68.5 mS/cm, V=2.25 L). The reaction volume was concentrated from 2.25 L to 1 L (conductivity=76.7 mS/cm, V=1 L; conductivity=74.2 mS/cm, V=3.5 L). RO filtration in dialfiltration mode began (always 1 L of retentate in tank). After each liter of permeate was collected, a conductivity measurement from the permeate flow was made. After 4 L of permeate was collected, a sample was collected.

### Example 3

### Procedure for Synthesizing an Array of 72 Polyionenes Using Dimorpholinos or Dipiperidines and Dibromides

Stock solutions (3.2M) of six different dimorpholinos and dipiperidines and 12 different dibromides, all of which are shown below, were prepared in DMF. One-half ml of each sample was added to 8 ml tared and labeled vials, which were set up in an 8×12 array. Materials that would not dissolve in DMF were added neat to their appropriate vials (1.6 mmol) and 200 microliters of DMF was added as well. The samples were placed on a heater/shaker block at a temperature of 50° C. and shaken for 7 days. After four days, the temperature was turned up to 65° C. 200 microliters of deionized water was added to each sample after five days. After 7 days of heating and shaking, the samples were cooled to room temperature and precipitated with ether. Approximately 3 ml of ether were added to each sample. The sample was vortexed and allowed to settle. The ether was decanted off. This precipitation procedure was repeated a total of 3 times. The samples were air dried for about 17 hours then dissolved in water. A small piece of 500 molecular weight cut-off (MWCO) dialysis membrane was placed on the top of the vial. An open top screw cap was put on the vial to secure the membrane in place. The samples were placed upside down resting on a cap into a 100 ml graduated cylinder filled with deionized water. The water was changed each day for a week. The samples were then dried on the speed vacuum at 45° C. for approximately 48 hours. A yield was obtained. Solutions of the samples were prepared at 10 mg/ml and 1 mg/ml in water.

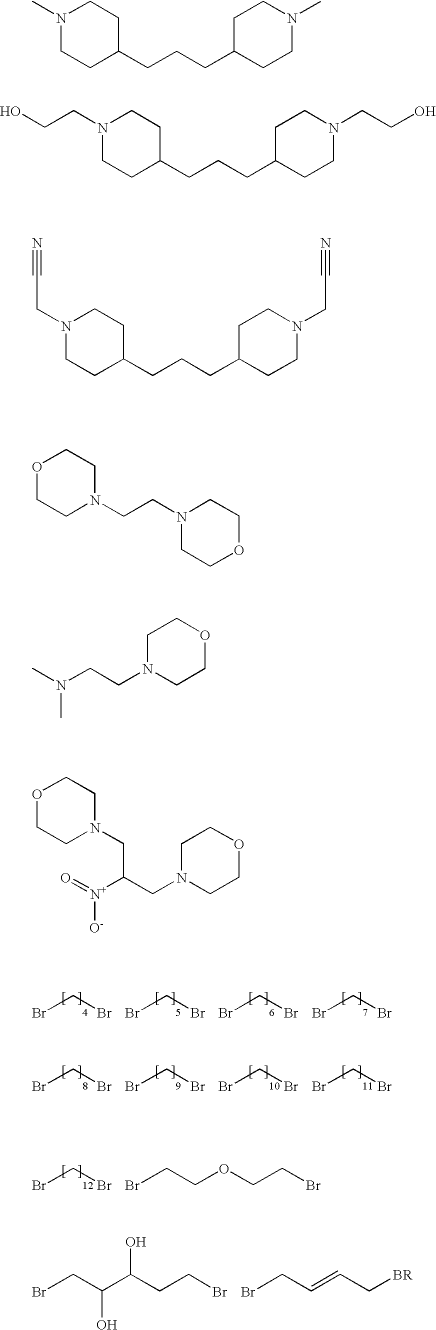

### Example 4

### Procedure for Synthesizing an Array of 96 Polyionenes Using Dianilines and Diepoxides

Stock solutions (3.2M) of 12 different diepoxides, which are shown below, were prepared in DMF. One-half ml of each sample was added to 8 ml tared and labeled vials which were set up in an 8×12 array. The 8 different dianiline materials, which are also shown below, were added neat to their appropriate vials (1.6 mmol) and 1 molar equivalent of acetic acid was added to each sample, as well as 250 microliters of DMF. The samples were placed on a heater/shaker block at room temperature and shaken for 7 days. One molar equivalent of acetic acid was added after ˜24 hours. Two more molar equivalents of acetic acid were added after 48 hours. After 72 hours, the heat was turned up to 50° C. and another equivalent of acetic acid was added to any sample that was not fully dissolved. After 7 days of heating/shaking the samples were cooled to room temperature and precipitated with ether. Approximately 3 mls of ether were added to each sample. The sample was vortexed and allowed to settle. The ether was decanted off. This precipitation procedure was repeated a total of 3 times. The samples were air dried for approximately 17 hours then dissolved in water. A small piece of 500 molecular weight cut-off (MWCO) dialysis membrane was placed on the top of the vial. An open top screw cap was put on the vial to secure the membrane in place. The samples were placed upside down resting on a cap into a 100 ml graduated cylinder filled with deionized water. The water was changed each day for a week. The samples were then dried on the speed vacuum at 45° C. for approximately 48 hours. A yield was obtained. Solutions of the samples were prepared at 10 mg/ml and 1 mg/ml in water.

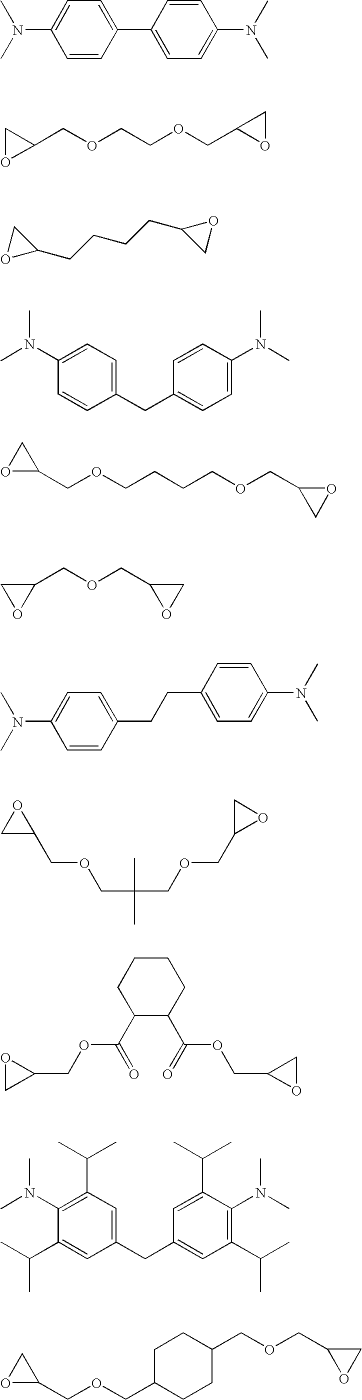

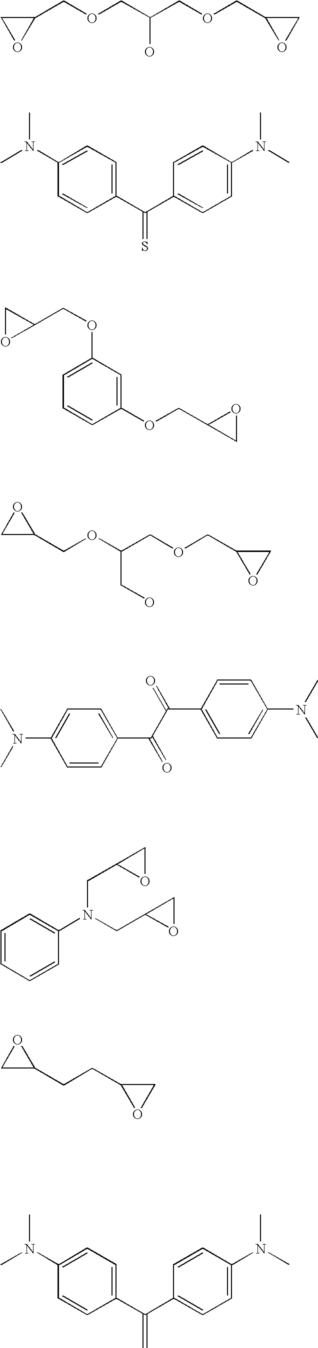

In Examples 5-20 below, oligomers can be prepared by dissolving the polymer and oligomer mixture in water or another appropriate solvent. After the mixture is dissolved, it is filtered through a membrane of the lowest molecular weight desired (e.g., 1 kDa). The retentate from the first step is then filtered through a second membrane of the highest molecular weight desired (e.g., 2 kDa, 3 kDa) and the filtrate contains the product oligomers.

Alternatively, the order of the membrane filtration can be reversed. Under these circumstances, the polymer and oligomer mixture is filtered through a membrane of the highest molecular weight desired (e.g., 2 kDa, 3 kDa). The filtrate from the first step is retained and is then filtered through a membrane of the lowest molecular weight desired (e.g., 1 kDa). The retentate of the second step contained the desired oligomers.

### Example 5

### Preparation of poly(hexamethylenebiscyanoguanidine-alt-4,9-dioxadodecane)

Hexamethylenebiscyanoguanidine (3.99 mmoles, 1.00 g) and 4,9-dioxa-1,12-dodecanediamine (3.99 mmoles, 0.848 ml) were added to a 40 ml vial with a septa-cap followed by 2 equivalents of concentrated HCl. The mixture was heated to 135-145° C. in a shaker overnight. A clear yellow, brittle solid was obtained.

### Example 6

### Preparation of poly(4,4′-trimethylenebis(1-methylpiperidinium)-alt-octane)

4,4′-Trimethylenebis(1-methylpiperidine)-alt-1,8-Dibromooctane was prepared by dissolving 4,4′-Trimethylenebis(1-methylpiperidine) (39.9 ml) in 30 ml of DMF in a 250 ml Erlenmeyer flask. 1,8-Dibromooctane (27.63 ml) was also added to the flask. The reaction was purged with nitrogen, covered with a septum, and stirred with a magnetic stir plate. The initial solution was clear. After approximately 20 minutes of stirring the reaction exothermed and solidified. A light yellow solid polymer formed and was left to further polymerize for a week.

### Example 7

### Preparation of poly(4-(dimethylamino)phenyldiphenylphosphonium-alt-dodecane)

4-(Dimethylamino)phenyldiphenylphosphine (1.73 mmoles, 0.529 g) and 1,12-dibromododecane (1.73 mmoles, 0.569 g) were dissolved in DMF (1 ml) and shaken for 1 week.

### Example 8

### Preparation of poly(4,4′-trimethylenedipyridinium-alt-hexane)

4,4′-Trimethylenedipyridine (3.46 mmoles, 0.687 g) was added to a 40 ml vial followed by 2.3 ml of DMF/methanol (1:1 v:v). 1,6-dibromohexane (3.46 mmoles, 0.533 ml) was added and the vial was capped with a septa-cap. The vial was purged with nitrogen and placed in a shaker for 1 week.

### Example 9

### Preparation of poly(4,4′-trimethylenedipyridinium-alt-nonane)

4,4′-Trimethylenedipyridine (3.46 mmoles, 0.687 g) was added to a 40 ml vial followed by 2.3 ml of DMF/methanol (1:1 v:v). 1,9-dibromononane (3.46 mmoles, 0.705 ml) was added and the vial was capped with a septa-cap. The vial was purged with nitrogen and placed in a shaker for 1 week.

### Example 10

### Preparation of poly(N,N-dimethylpropylammonium-alt-N,N-dimethylhexylammonium)

N,N,N′,N′-Tetramethyl-1,3-propanediamine-alt-1,6-Dibromohexane was prepared by dissolving N,N,N′,N′-Tetramethyl-1,3-propanediamine (31.9 ml) in 40 ml of DMF in a 250 Erlenmeyer flask. 1,6-Dibromohexane (29.3 ml) was added to the flask. The reaction was purged with nitrogen, covered with a septum, and stirred with a magnetic stir plate. The initial solution was clear. A very quick reaction that exothermed and solidified occurred. An off white solid polymer formed and was left to further polymerize for a week.

### Example 11

### Preparation of poly(hexamethylene biscyano guanidine-alt-nonane)

Hexamethylenebiscyanoguanidine (3.99 mmoles, 1.00 g) and 1,9-diaminononane (3.99 mmoles, 0.623 g) were added to a 40 ml vial with a septa-cap followed by 2 equivalents of concentrated HCl. The mixture was heated to 135-145° C. in a shaker overnight.

### Example 12

### Preparation of poly(4,4′-trimethylenedipiperidinium-alt-hexane)

4,4′-Trimethylenedipiperidine (3.466 mmoles, 1.139 g) was added to a 40 ml vial followed by 2 ml DMF/MeOH (1:1 v:v). 1,6-Dibromohexane (3.466 mmoles, 0.533 ml) was added and the vial was capped with a septa-cap. The vial was purged with nitrogen and placed in a shaker for 1 week.

### Example 13

### Preparation of poly(hexamethylenebiscyanoguanidine-alt-hydrazine)

Hexamethylene biscyano guanidine (4.00 mmoles, 1.00 g) and hydrazine (4.00 mmoles, 0.274 g) were added to a 40 mL vial with a septa-cap followed by 2 equivalents of concentrated HCl. The mixture was heated to 165° C. in an oil-bath for 3 hours. A pink foam was obtained.

### Example 14

### Preparation of poly(4-(dimethylamino)phenyldiphenylphosphonium-alt-nonane)

4-(Dimethylamino)phenyldiphenylphosphine (1.73 mmoles, 0.529 g) and 1,9-dibromononane (1.73 mmoles, 0.352 g) were dissolved in DMF (1 ml) and shaken for 1 week.

### Example 15

### Preparation of poly(4-(dimethylamino)phenyldiphenylphosphonium-alt-decane)

4-(Dimethylamino)phenyldiphenylphosphine (1.73 mmoles, 0.529 g) and 1,10-dibromodecane (1.73 mmoles, 1.04 g) were dissolved in DMF (1 ml) and shaken for 1 week.

### Example 16

### Preparation of poly(hexamethylene biscyano guanidine-alt-1,3-aminoguanidine)

Hexamethylene biscyano guanidine (4.00 mmoles, 1.00 g) and 1,3-aminoguanidine (4.00 mmoles, 0.502 g) were added to a 40 ml vial with a septa-cap followed by 2 equivalents of concentrated HCl. The mixture was heated to 165° C. in an oil-bath for 3 hours. An orange solid was obtained.

### Example 17

### Preparation of poly(1,3-bis(diphenylphosphonium)propane-alt-butane)

1,3-Bis(diphenylphosphino)propane (1.33 mmoles, 0.550 g) and 1,4-dibromobutane (1.33 mmoles, 0.159 g) were dissolved in DMF (0.769 ml) and shaken for 1 week.

### Example 18

### Preparation of poly(4-(dimethylamino)phenyldiphenylphosphonium-alt-butane)

4-(Dimethylamino)phenyldiphenylphosphine (1.73 mmoles, 0.529 g) and 1,4-dibromobutane (1.73 mmoles, 0.207 g) were dissolved in DMF (1 ml) and shaken for 1 week. A viscous liquid was obtained.

### Example 19

### Preparation of poly(1,4-bis(diphenylphosphonium)butane-alt-butane)

1,4-Bis(diphenylphosphino)butane (2.31 mmoles, 0.986 g) and 1,4-dibromobutane (2.31 mmoles, 0.276 g) were dissolved in DMF (1.333 ml) and shaken for 1 week. A viscous liquid was obtained.

### Example 20

### Preparation of poly(trimethylenedipyridinium-alt-2,7-dihydroxyoctane)

Trimethylenedipyridine (100 g) was placed in a roundbottom flask. To the flask was added 1,2,7,8-diepoxyoctane (71.72 g). The reaction was stirred under nitrogen at room temperature for 20 min. until nearly all the trimethylenedipyridine was dissolved. At this time, acetic acid (121 g) was slowly added dropwise over a 24 hour period. The reaction was stirred at room temperature for an additional four days. The resulting solid material was dark blue and highly viscous.

### Example 21

### Relationship Between Cytotoxicity and Molecular Weight

Poly(trimethylene dipyridine-alt-octane) (TMDP-C₈) and poly(trimethylene dipyridine-alt-2,7-dihydroxyoctane) (TMDP-C₈(OH)₂) differ significantly in their cytotoxicity to mammalian cells in vitro. The polymers were fractionated by ultra filtration after synthesis into different MW groups. Serial 2-fold dilutions of polymer were plated on tissue culture plates containing confluent Vero or A-549 cells. Plates were incubated 24 hr, then assayed for metabolic activity.

The oligomers were screened for in vitro cytotoxicity following 18-24 hr exposure using the metabolic indicator Alamar Blue™ (Alamar Biosciences, Sacramento, Calif.) against the A-549 lung epithelial cell line according to the manufacturer's instructions. Briefly, flat-bottom polystyrene microtiter plates were seeded with 2×10⁴ cells in Ham's F-12 medium (Invitrogen) containing 10% fetal bovine serum (FBS) and were incubated ˜48 hr at 37° C., until cells were confluent. Medium was then aspirated off and replaced with medium lacking FBS. The oligomers were added to the first row (final concentration 5 mg/ml) and serial 2-fold dilutions were prepared across the plate. Plates were incubated 18-24 hr at 37° C.; medium containing test compounds was aspirated off, wells were washed once with PBS and fresh medium (lacking FBS and phenol red) was added. Alamar Blue™ was added (final concentration 30%); cells were incubated 4 hr and absorbance was read at 570 nm. The IC₅₀ was then calculated.

As overall molecular weight decreased, the IC₅₀ increased (i.e, becomes less toxic) as shown in Table 1.

TABLE 1
IC₅₀ (μg/ml) against A-549 or Vero Cells (Alamar Blue Assay):

| Description | IC₅₀ |
|---|---|
| TMDP-C₈(OH)₂ ˜1-2 kDa | >5000 |
| TMDP-C₈(OH)₂ 1-3 kDa | 400 |
| TMDP-C₈(OH)₂ >3 kDa | 50 |
| TMDP-C₈ 1-3 kDa | 1484 |
| TMDP-C₈ 3-5 kDa | 106 |
| TMDP-C₈ 5-10 kDa | 31 |
| TMDP-C₈ >10 kDa | 26 |

### Example 22

Histological Effects of Varying Oligomer Molecular Weight

Three different MW fractions of poly(trimethylene dipyridine-alt-2,7-dihydroxyoctane) (TMDP-C₈(OH)₂) were tested in non-GLP 4-day toxicity studies in rats. Seventy-five, 150, 250 and 500 mg/kg/day doses of the less than 1 kDa, 1-3 kDa or greater than 3 kDa oligomers were administered to groups of 5 rats by oral gavage over 4 days. Animals were euthanized on Day 5, the G.I. tract, liver, kidney spleen and heart were processed and examined for histopathology. For the less than 1 kDa (as prepared from the filtrate from passing an oligomer-containing solution through a 1 kDa cutoff membrane) and 1-3 kDa compounds, cecal mucosal hyperplasia (described as “minimal”) constituted the only finding, and only at the highest doses (500 mg/kg). Similar findings have been observed when substances such as starches are incompletely absorbed in the small intestine. Notwithstanding, in the case of the greater than 3 kDa material (the retentate from passing an oligomer-containing solution through a 3 kDa cutoff membrane), hyperplasia was also reported for the colon.

### Example 23

### Tolerance of Oligomers in Lungs

A preliminary study was conducted to examine the relationship between cytotoxicity assayed in vitro and toxicity in vivo. Four concentrations of 3 different, but related, oligomers were examined in 16 groups of 4 animals (an additional group received saline as a control). Animals were anesthetized and received a single 100 μl dose of oligomer into the left lung. Concentrations tested included 0.1, 1, 10, and 20 mg/ml, corresponding respectively to approximately 20×, 200×, 2000×, and 4000× the in vitro MIC against P. aeruginosa. Compounds included 2 different MW fractions (less than 1 kDa; 1-3 kDa) of the chloride salt of poly(trimethylene dipyridine-alt-octane) (TMDP-C₈) and a 1.14 kDa pentamer synthesized with the same starting materials as TMDP-C₈. In vitro IC₅₀ values for these compounds are shown in Table 2.

TABLE 2
In vitro Cytotoxicity of Compounds Tested in First Rat Lung Study

| Compound | Mol Wt. | | IC₅₀ |
|---|---|---|---|
| TMDP-C₈ >1k | >1 | kDa | 30 μg/ml |
| TMDP-C₈ 1-3k | 1-3 | kDa | 322 μg/ml |
| TMDP-C₈ 1.14k | 1.14 | kDa | 3800 μg/ml |

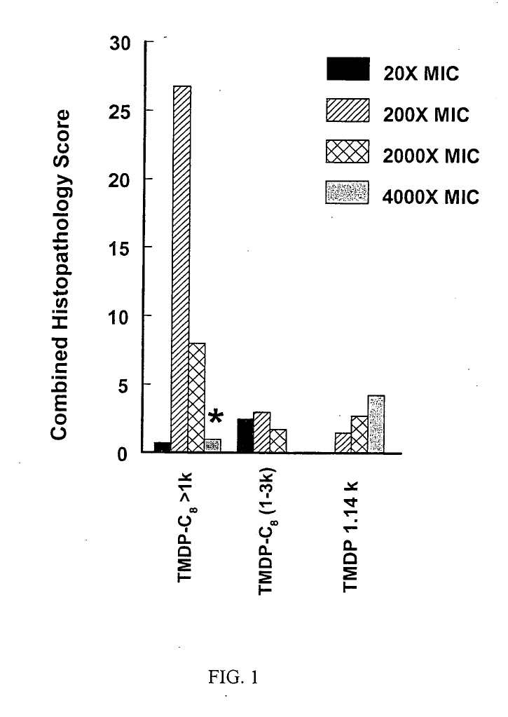

Following instillation, animals were monitored for survival, appearance, food and water intake and body weight change over 72 hr, then were euthanized. Lungs were excised, processed for hematoxylin-eosin staining and were examined and scored for evidence of pneumonia, alveolar edema and lymphoid hyperplasia. Scoring for each histopathological finding ranged from 1 (minimal) to 4 (severe). For each animal and drug concentration, 5 different areas were scored. To facilitate comparison between dosages of different compounds, scores for histopathology were combined for each animal (theoretical total of 60). The data are summarized in FIG. 1.

Histopathology was more severe for animals receiving the high molecular weight oligomer. This compound is also the most cytotoxic (lowest IC₅₀) when tested in vivo (Table 2), and indeed 2 rats died within 24 hr of administration at the highest dose (2 mg in 100 μl). It is important to note, however, that this represents a dose that is 4000× the MIC of the compound in vitro. For the lower molecular weight species, histopathology appeared fairly modest even at high multiples of the MIC (2,000-4000-fold), and this is generally in agreement with the in vitro cytotoxicity results.

### Example 24

### Antimicrobial Activity of Polyionenes

Both about 1-10 kDa fractions of poly(trimethylene dipyridine-alt-octane chloride) (TMDP-C₈) and about 1.2 kDa poly(trimethylene dipyridine-alt-2,7-dihydroxyoctane chloride) (TMDP-C₈(OH)₂) have broad, though not identical antimicrobial activity. They have been tested extensively against ˜50 different strains of bacteria and Candida spp., and against most strains, minimum inhibitory concentrations (MIC's) do not vary by more than 2-fold (the limit of reproducible accuracy of the MIC broth dilution assay). Some representative activities are shown in Tables 3, 4 and 5.

TABLE 3
MIC (μg/ml) Against Selected Pathogens

| | Streptococcus | E. coli | Candida | | P. aeruginosa | |
|---|---|---|---|---|---|---|
| | spp. (9) | (2) | spp. (8) | A. baumannii | (5) | B. cepacia |
| TMDP- | 0.2-6.25 | 0.8 | 0.4-3.0 | 3.12 | 1.6-3.2 | 6.4 |
| C₈ | | | | | | |
| TMDP- | 0.4-2500 | 0.8 | 0.4-3.0 | 2500 | 1.6 | 3200 |
| C₈(OH)₂ | | | | | | |

(Numbers in parentheses indicate numbers of species/strains tested.)

TABLE 4
Susceptibility of Bacteria to Polyionenes

| | | MIC (μg/mL) | |
|---|---|---|---|
| | | TMDP- | |
| Organism | Strain ID | C₈(OH)₂ | TMDP-C₈ |
| Acinetobacter baumannii | U.S. Army | 50 | 3.2 |
| | Inst. Surg. Res. | | |
| | 980112001 | | |
| Aeromonas hydrophila | ATCC 35654 | 1.6 | 1.6 |
| Branhamella catarrhalis | ATCC 25238 | 0.05 | 0.2 |
| Burkholderia cepacia | ATCC 25416 | >50 | 6.3 |
| Corynebacterium diphtheriae | ATCC 27010 | 0.05 | 0.1 |
| Enterococcus faecalis | ATCC 29212 | 0.05 | 0.4 |
| Enterococcus faecium | ATCC 19434 | 0.05 | 0.8 |
| Escherichia coli | ATCC 25922 | 1.6 | 0.8 |
| Haemophilus influenzae | ATCC 33391 | 6.3 | 1.6 |
| Klebsiella pneumoniae | ATCC 13883 | 1.6 | 1.6 |
| Listeria innocua | ATCC 33090 | 0.4 | 0.4 |
| Neisseria meningitidis | ATCC 13077 | 0.2 | 0.4 |
| Neisseria mucosa | ATCC 19695 | 0.8 | 0.1 |
| Neisseria sicca | ATCC 9913 | 0.1 | 0.8 |
| Neisseria subflava | ATCC 49275 | 0.1 | 0.1 |
| Pseudomonas aeruginosa | ATCC 27853 | 1.6 | 1.6 |
| Proteus mirabilis | U.S. Army | 6.3 | 6.3 |
| | Inst. Surg. Res. | | |
| | 770822034 | | |
| Rhodococcus equi | ATCC 6939 | 0.8 | 0.8 |
| Serratia marcescens | ATCC 13880 | 1.6 | 1.6 |
| Staphylococcus aureus | ATCC 29213 | 0.2 | 0.8 |
| Streptococcus aureus | C946, Univ. | 0.05 | ND |
| | Brit. Columbia | | |
| Staphylococcus epidermidis | ATCC 14990 | 0.1 | 0.4 |
| Staphylococcus epidermidis | Genzyme | 0.05 | 0.4 |
| | Corp. 14990 | | |
| Staphylococcus hominis | ATCC 27844 | 0.05 | 0.1 |
| Stenotrophomonas maltophilia | ATCC 13637 | 0.8 | 0.8 |
| Streptococcus agalactiae | ATCC 49446 | 1.6 | 0.8 |
| Streptococcus Group D | ATCC 12959 | 6.31 | 0.8 |
| Streptococcus mitis | ATCC 6249 | >50 | 1.6 |
| Streptococcus mitis | ATCC 49456 | 12.5 | 1.6 |
| Streptococcus mutans | ATCC 25175 | 0.1 | 0.2 |
| Streptococcus oralis | ATCC 9811 | >50 | 3.2 |
| Streptococcus oralis | ATCC 55229 | >50 | 1.6 |
| Streptococcus pneumoniae | ATCC 33400 | 12.5 | 1.6 |
| Streptococcus pyogenes | ATCC 12344 | 0.4 | 0.2 |
| Streptococcus salivarius | ATCC 13419 | 0.1 | 0.4 |
| Streptococcus sanguis | ATCC 10556 | <0.05 | 0.1 |

TABLE 5
Susceptibility of Candida Species to Polyionenes

| | Strain | MIC (μg/mL) | |
|---|---|---|---|
| Organism | Identification | TMDP-C₈(OH)₂ | TMDP-C₈ |
| Candida albicans | ATCC 18804 | 0.4 | 0.1 |
| Candida albicans | 4090, Univ. TX | 0.4 | 0.4 |
| | Hlth. Sci. Ctr. | | |
| | San Antonio | | |
| | (UTHSCSA) | | |
| Candida albicans | 4111, UTHSCSA | 0.4 | 0.4 |
| Candida albicans | 4227, UTHSCSA | 0.8 | 0.2 |
| Candida albicans | 4507, UTHSCSA | 0.8 | 0.4 |
| Candida dublinensis | 4116, UTHSCSA | 0.4 | 0.4 |
| Candida glabrata | ATCC 90030 | 0.4 | 0.4 |
| Candida glabrata | 4233, UTHSCSA | 0.4 | 0.8 |
| Candida glabrata | 4760, UTHSCSA | 0.4 | 0.4 |
| Candida glabrata | 4758, UTHSCSA | 0.4 | 0.4 |
| Candida krusei | ATCC 2340 | 0.2 | 0.2 |
| Candida krusei | 4566, UTHSCSA | 0.2 | 0.4 |
| Candida krusei | 4835, UTHSCSA | 0.2 | 0.4 |
| Candida lusitaniae | ATCC 34449 | 0.1 | 0.4 |
| Candida lusitaniae | ATCC 42720 | 0.2 | 0.4 |
| Candida parapsilosis | 6196, UTHSCSA | 0.2 | 0.2 |
| Candida tropicalis | ATCC 1369 | 0.1 | 0.4 |
| Candida tropicalis | 4305, UTHSCSA | 0.2 | 0.4 |

### Example 25

Susceptibility of Antibiotic-Resistant Bacteria to Polyionenes

About 1-10 kDa fractions of TMDP-C₈ and about 1.2 kDaTMDP-C₈(OH)₂ have also been tested against bacterial strains resistant to conventional antibiotics. Antimicrobial activity against methcillin-resistant S. aureus, vancomycin-resistant Enterococcus spp., glycopeptide-resistant S. aureus and multiply resistant P. aeruginosa, Stenotrophomonas maltophilia and Acinetobacter spp. were within a 2-fold dilution of those obtained using antibiotic-susceptible strains of the same species (data not shown). This suggests that mechanisms of action of antimicrobial polymers differ from those of conventional antibiotics.

### Example 26

### Development of Antibiotic Resistance

We have performed in vitro resistance selection studies using 4 different classes of antimicrobial polymers (about 1-10 kDa biguanides, about 1-10 kDa phosphonium ionenes, dipiperidine ionenes (about 1-10 kDa TMBDP-C₈ and about 1.2 and 5 kDa dipyridine ionenes) against 5 bacterial strains (E. coli (ATCC 25922 or ATCC 11775), E. faecium (ATCC 19434) E. faecalis (ATCC 29212), S. aureus (ATCC 29213) or P. aeruginosa (ATCC 27853)). To select for resistance, ATCC strains were passaged 20-25 times in vitro in the presence of sub-inhibitory concentrations of the selecting compound. Isolates from each passage were then tested in an MIC assay for susceptibility to the selecting compound, to related non-peptide antimicrobial polymers and to conventional antibiotics. For these studies, resistance was defined as a change of ≧4-fold in the MIC.

Results from resistance evolution studies showed that for the biguanide and phosphonium ionene classes, there was no change in susceptibility over 25 passages for S. aureus, E. coli. P. aeruginosa or E. faecium. For TMBDP-C₈ (S. aureus) and TMDP-C₈ (OH)₂, (P. aeruginosa) resistance emerged after 14-16 passages (in independent studies). This time course of resistance evolution is comparable to that seen for antimicrobial peptides, although published studies were run for only 7-15 passages. For comparison, those studies also examined resistance evolution to norfloxacin and gentamicin. Against P. aeruginosa, the MIC of norfloxacin rose 10-fold and that of gentamicin 190-fold within 11 passages. Against MRSA, the MIC of norfloxacin rose 85-fold over 15 passages.

### Example 27

### Activity of Ionenes in Infected Wounds

The non-substituted C₈-containing compound TMDP-C₈ (about 1-10 kDa) was tested at 10 mg/ml for its ability to reduce S. aureus infections introduced into dermal wounds in pigs. Compared with controls, treatment reduced recoverable microbial load by 4 logarithmic units; in this model, reduction by ≧1 log is considered significant. In parallel studies examining wound healing (in the absence of introduced microbial infection), treatment with TMDP-C₈ did not inhibit or retard healing. These 2 preliminary studies suggest that the cationic compound retains antimicrobial activity in the context of tissue/tissue exudate, and that at least when topically applied, does not appear to inhibit wound healing.

### Example 28

### Lowering of Oral Microbial Load

Studies were also conducted to assess the effect of treatment using about 1.2-9 kDa TMDP-C₈(OH)₂ on oral microbial load in a hamster model of irradiation-induced oral mucositis. Following irradiation, animals were dosed 3× daily into the left cheek pouch with 15 mg/kg TMDP-C₈(OH)₂. Microbial samples were collected one hour after the final treatment on Days 8, 14 and 20 following irradiation, corresponding to different phases of disease. At each time point, total microbial load was reduced by approximately 1-2 logarithmic units. (In this model, this dose administration was shown to reduce ulceration by ˜80%).

### Example 29

### Antimicrobial Activity of Polyionenes in Sputum from CF Patients

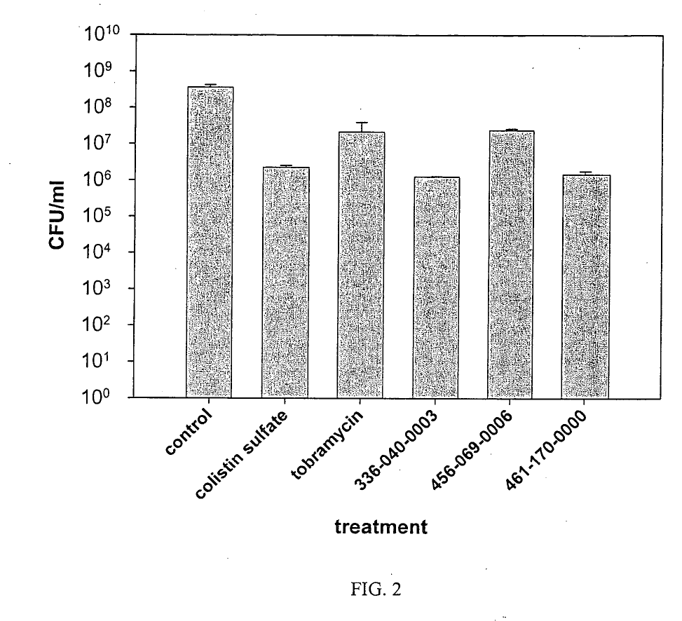

The methods used in this example were modified from those of Sajjan and coworkers (U. Sajjan, et al., “P113D, An antimicrobial peptide active against Pseudomonas aeruginosa, retains activity in the presence of sputum from cystic fibrosis patients,” Antimicrobial Agents and Chemotherapy 45(12): 3437-3444). Briefly, sputum specimens from 5 cystic fibrosis (CF) patients was pooled, aliquotted at 250 μl, and stored at −80° C. until use. For each experiment, sputum was thawed, was diluted 1:10 in 0.85% sterile saline and was incubated for 1 hr at 35° C. in the presence of 100 μg/ml Domase Alpha. Test compounds were then added to a final concentration of 100× the Minimum Inhibitory Concentration (range: 100-600 μg/ml), and samples were incubated for 6 hr at 35° C. Ten-fold serial dilutions were then prepared, plated on Tryptic soy agar medium, incubated for 48 hr at 35° C., and colonies were enumerated. The results are shown in FIG. 2. Colistin sulfate and tobramycin, antibiotics commonly used to treat CF patients, are included for comparison. The three polyionenes tested, poly(TMDP-C₈), a 3.5-mer of TMDP-C₈ and poly(trimethylene dipyridine-alt-5-oxanonane), exhibited antimicrobial activity similar to that of the two common antibiotics.

In separate studies, this pool of patient sputum specimens was shown to contain Pseudomonas aeruginosa, Alcaligenes faecalis, Acinetobacter haemolyticus and CDC group VB3, in addition to other uncharacterized species.

### Example 30

### Antimicrobial Activity of Polyionenes in Rat CF Models

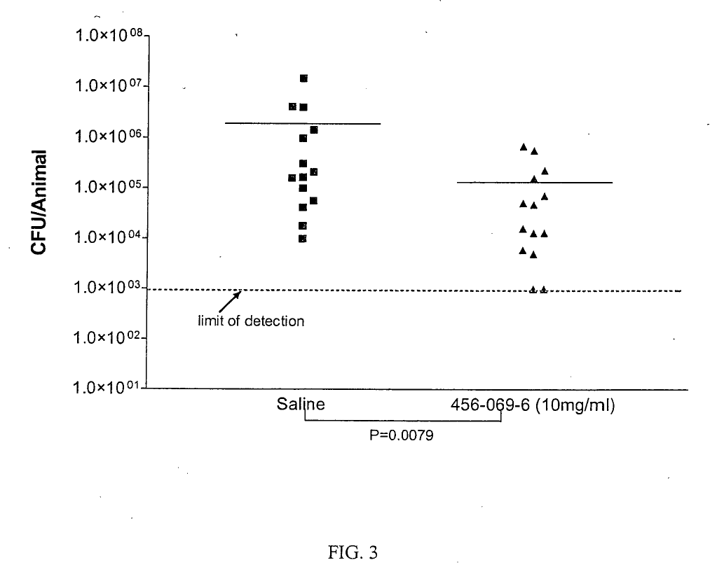

Male Sprague-Dawley rats were inoculated with 7×10⁴ CFU Pseudomonas aeruginosa by intratracheal instillation into the lungs, according to the model developed by Hash and coworkers (Cash, H. A., et al., “A rat model of chronic respiratory infection with Pseudomonas aeruginosa,” Am. Rev. Respir. Dis. 119:453-459 (1979). Briefly, P. aeruginosa was embedded in agarose beads of approximately 30 micron diameter in a volume of 100 μl for the inoculum. A 10 mg/mL solution of poly(trimethylene dipyridine-alt-5-oxanonane (indicated as 456-069-6 in FIG. 3) or saline was administered daily by intranasal delivery of 100 μl of one of the solutions on days 3 through 6 post-infection. Bacterial load was determined by serial dilution and culture of lung homogenates on day 6. The results are shown in FIG. 3. The arithmetic mean for colony forming units in rats receiving saline was 1.89×10⁶, while rats receiving the polyionene had an arithmetic mean of 1.35×10⁵ CFUs. A similar reduction was seen in the geometric means of CFUs among the rat population treated with the polyionene, from 2.87×10⁵ to 2.82×10⁴. The difference in number of CFUs among treatment groups was significant, with a p value of 0.0079.

### Example 31

### Safety and Efficacy of Poly(trimethylene dipyridine-alt-5-oxanonane)

Poly(trimethylene dipyridine-alt-5-oxanonane) having a molecular weight 0.5-1 kDa was tested in A-549 lung epithelial cells to determine its IC₅₀ value in comparison with tobramycin, a conventional antibiotic. In addition, the minimum inhibitory concentrations for the two compounds were determined in vitro for a number of bacterial strains and species. The results are as shown below, where all concentrations are expressed as μg/mL:

| | | | P. aeruginosa | P. aeruginosa | B. | S. |
|---|---|---|---|---|---|---|
| Compound | IC₅₀ | S. aureus | G38 | PA01 | cepacia | maltophilia |
| Tobramycin | 5000 | 0.195 | 0.39 | 0.39 | >50 | 25 |
| Polyionene | 1849 | 0.78 | 6.25 | 3.125 | >50 | 0.78 |

The polyionene was about three times less toxic than the antibiotic and had acceptable antimicrobial activity.

While this invention has been particularly shown and described with references to preferred embodiments thereof, it will be understood by those skilled in the art that various changes in form and details may be made therein without departing from the scope of the invention encompassed by the appended claims.

What is claimed is:

1. A composition comprising a compound represented by Structural Formula (I): 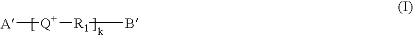
or a salt thereof, wherein:
-A′ is —R₁′ or —R₁-Q;
—B′ is -Q⁺-R₁′ or -Q;
each —R₁— is a substituted hydrocarbylene and is independently selected;
—R₁′ is a substituted or unsubstituted hydrocarbyl group optionally interrupted with one or more heteroatoms; and
each Q⁺ is independently represented by a structural formula selected from: 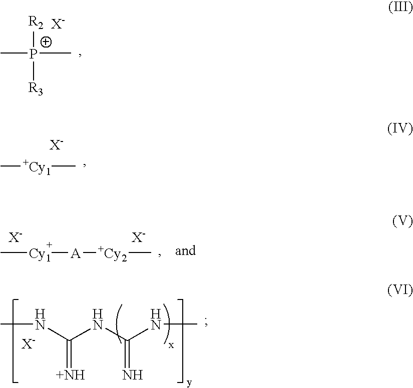
-Q is represented by a structural formula selected from: 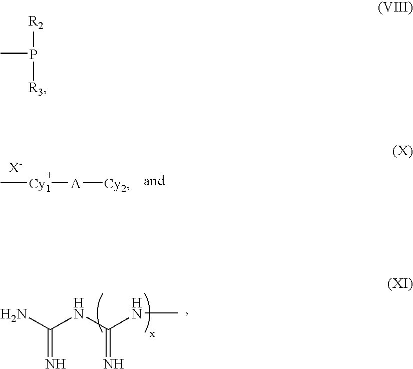
wherein tertiary phosphorus atoms of Structural Formula (VIII), tertiary nitrogen atoms of Structural Formulas (IX) and (X) and primary nitrogen atoms of Structural Formula (XI) are optionally alkylated or protonated;
each Cy₁⁺ and Cy₂⁺ is independently a quaternary nitrogen-containing monocyclic heteroaromatic ring, a protonated tertiary nitrogen-containing non-aromatic heterocyclic ring or a quaternary nitrogen-containing non-aromatic ring;
each Cy₁ and Cy₂ is independently a nitrogen-containing non-aromatic heterocyclic ring or a nitrogen-containing heteroaromatic ring;
A is a covalent bond, or a substituted or unsubstituted lower alkylene group;
R₂ and R₃ are independently —H or a substituted or unsubstituted aliphatic or aromatic group;
each X⁻, separately or taken together with other X⁻s, is an anion;
k is an integer from 1 to 25;
x is an integer from 0-4; and
y is an integer from 1-5.

2. The composition of claim 1, wherein k is an integer from 1-15.

3. The composition of claim 2, wherein -Q⁺- is represented by the structural formula: 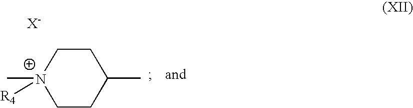
-Q is represented by the structural formula: 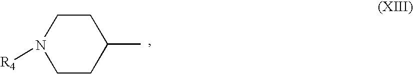
wherein R₄ is —H or a substituted or unsubstituted lower alkyl group.

4. The composition of claim 3, wherein R₄ is a lower alkyl or hydroxy substituted lower alkyl group.

5. The composition of claim 2, wherein -Q⁺- is represented by the structural formula: 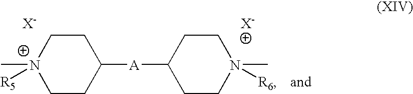
-Q is represented by the structural formula: 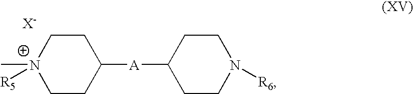
wherein A is a bond or substituted or unsubstituted lower alkylene group, and wherein R₅ and R₆ are each independently —H or a substituted or unsubstituted lower alkyl group.

6. The composition of claim 5, wherein R₅ and R₆ are each an alkyl group or a hydroxyalkyl group.

7. The composition of claim 6, wherein A is an unsubstituted straight chained lower alkylene group.

8. The composition of claim 7, wherein R₁ is a substituted straight chained lower alkylene group or polyalkylene glycol substituted with one or more —OH groups and R₁′ is a substituted or unsubstituted straight chained lower alkyl group or polyalkylene glycol, wherein the lower alkyl group or the polyalkylene glycol of R₁′ is optionally substituted with one or more —OH, leaving groups or oxiranyl groups.

9. The composition of claim 2, wherein -Q⁺- is represented by the structural formula: 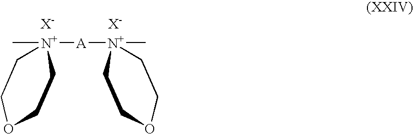
and -Q is represented by the structural formula:

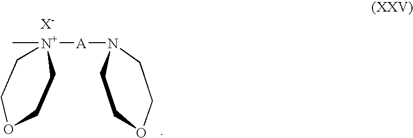

10. The composition of claim 2, wherein -Q⁺- is represented by the structural formula: 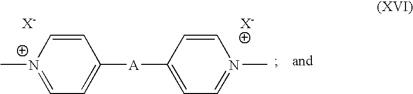
-Q is represented by the structural formula: 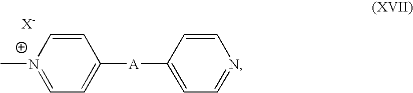
wherein A is a bond or a substituted or unsubstituted lower alkylene or lower alkylene glycol group.

11. The composition of claim 10, wherein A is an unsubstituted straight chained lower alkylene group.

12. The composition of claim 11, wherein R₁ is a substituted straight chained lower alkylene group or polyalkylene glycol substituted with one or more —OH groups and R₁′ is a substituted or unsubstituted straight chained lower alkyl group or polyalkylene glycol, wherein the lower alkyl group or the polyalkylene glycol of R₁′ is optionally substituted with one or more —OH, leaving groups or oxiranyl groups.

13. The composition of claim 12, wherein -Q⁺-R₁— is represented by the structural formula:

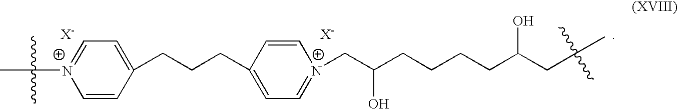

14. The composition of claim 2, wherein each -Q⁺- is represented by Structural Formula (III) or each -Q⁺- is represented by a structural formula independently selected from Structural Formula (II) and Structural Formula (III) and -Q is represented by Structural Formula (VII) or (VIII).

15. The composition of claim 2, wherein -Q⁺- is represented by Structural Formula (VI) and -Q is represented by Structural Formula (XI).

16. A pharmaceutical composition comprising a carrier or diluent and molecules represented by Structural Formula (I): 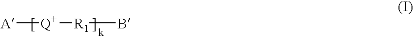
or a pharmaceutically acceptable salt thereof, wherein:
-A′ is —R₁′ or —R₁-Q;
—B′ is -Q⁺-R₁′ or -Q;
each —R₁— is independently a linker and is independently selected;
—R₁′ is a substituted or unsubstituted hydrocarbyl group optionally interrupted with one or more heteroatoms; and
each Q⁺ is independently represented by a structural formula selected from: 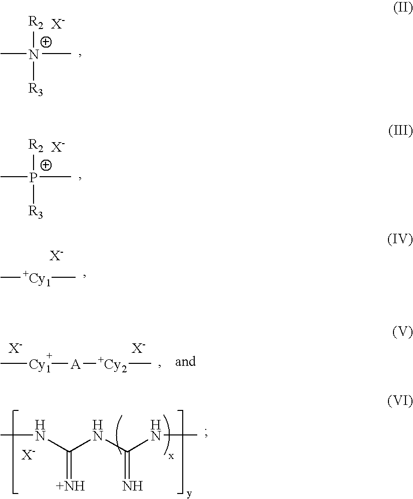
-Q is represented by a structural formula selected from: 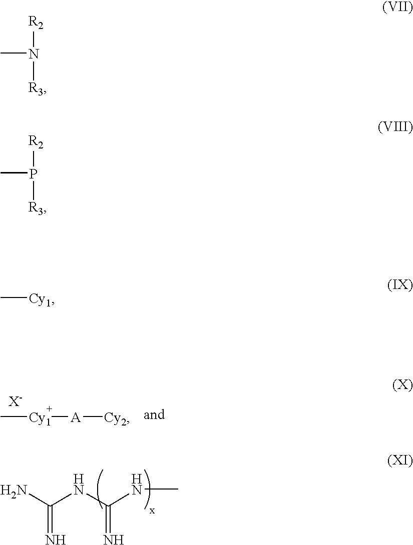
wherein tertiary phosphorus atoms of Structural Formula (VIII), tertiary nitrogen atoms of Structural Formulas (VII), (IX) and (X) and primary nitrogen atoms of Structural Formula (XI) are optionally protonated or alkylated;
each Cy₁⁺ and Cy₂⁺ is independently a quaternary nitrogen-containing monocyclic heteroaromatic ring, a protonated tertiary nitrogen-containing non-aromatic heterocyclic ring or a quaternary nitrogen-containing non-aromatic ring;
each Cy₁ and Cy₂ is independently a nitrogen-containing non-aromatic heterocyclic ring or a nitrogen-containing heteroaromatic ring;
A is a covalent bond, or a substituted or unsubstituted lower alkylene group;
R₂ and R₃ are independently —H or a substituted or unsubstituted aliphatic or aromatic group;
each X⁻, separately or taken together with other X⁻s, is a pharmaceutically acceptable anion;
k is an integer from 1 to 25;
x is an integer from 0-4; and
y is an integer from 1-5.

17. The pharmaceutical composition of claim 16, wherein k is an integer from 1-15.

18. The pharmaceutical composition of claim 17, wherein R₁ is a substituted or unsubstituted alkylene group and R₁′ is a substituted or unsubstituted alkyl group.

19. The pharmaceutical composition of claim 17, wherein each R₂ and R₃ are each independently an alkyl group or a hydroxyalkyl group.

20. The pharmaceutical composition of claim 19, wherein each -Q⁺- is represented by Structural Formula (II) and each -Q is represented by Structural Formula (VII).

21. The pharmaceutical composition of claim 20, wherein R₁ is a substituted or unsubstituted straight chained lower alkylene group or polyalkylene glycol, wherein the lower alkylene group or the polyalkylene glycol of R₁ is optionally substituted with one or more —OH groups and R₁′ is a substituted or unsubstituted straight chained lower alkyl group or polyalkylene glycol, wherein the lower alkyl group or the polyalkylene glycol of R₁′ is optionally substituted with one or more —OH, leaving groups or oxiranyl groups.

22. The pharmaceutical composition of claim 21, wherein R₁ is an unsubstituted straight chained lower alkylene group and R₁′ is an alkyl group substituted with a leaving group, wherein the leaving group is bromine, chlorine, or iodine.

23. The pharmaceutical composition of claim 17, wherein -Q⁺- is represented by the structural formula: 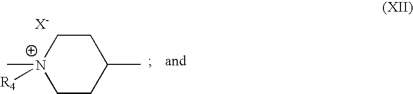
-Q is represented by the structural formula: 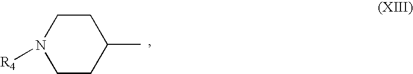
wherein R₄ is —H or a substituted or unsubstituted lower alkyl group.

24. The pharmaceutical composition of claim 23, wherein R₄ is a lower alkyl or hydroxy substituted lower alkyl group.

25. The pharmaceutical composition of claim 17, wherein -Q⁺- is represented by the structural formula: 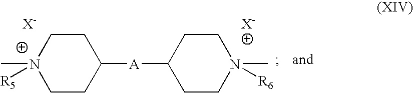
-Q is represented by the structural formula: 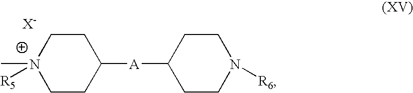
wherein A is a bond or substituted or unsubstituted lower alkylene group, and wherein R₅ and R₆ are each independently —H or a substituted or unsubstituted lower alkyl group.

26. The pharmaceutical composition of claim 25, wherein R₅ and R₆ are each an alkyl group or a hydroxyalkyl group.

27. The pharmaceutical composition of claim 26, wherein A is an unsubstituted straight chained lower alkylene group.

28. The pharmaceutical composition of claim 27, wherein R₁ is a substituted or unsubstituted straight chained lower alkylene group or polyalkylene glycol optionally substituted with one or more —OH groups and R₁′ is a substituted or unsubstituted straight chained lower alkyl group or polyalkylene glycol, wherein the lower alkyl group or the polyalkylene glycol of R₁′ is optionally substituted with one or more —OH, leaving groups or oxiranyl groups.

29. The pharmaceutical composition of claim 28, wherein R₁ is an unsubstituted straight chained lower alkylene group and R₁′ is an alkyl group substituted with a leaving group, wherein the leaving group is bromine, chlorine, or iodine.

30. The pharmaceutical composition of claim 28, wherein R₁ is an unsubstituted polyalkylene glycol or —CH₂CHOH(CH₂)ₙCHOHCH₂— wherein n is an integer from 0 to 8 and R₁′ is a polyalkylene glycol group substituted with an oxiranyl group or an alkylene group substituted with an oxiranyl group and a hydroxyl group.

31. The pharmaceutical composition of claim 17, wherein -Q⁺- is represented by the structural formula: 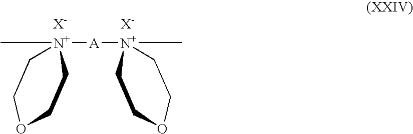
and -Q is represented by the structural formula:

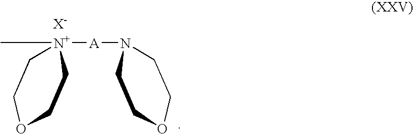

32. The pharmaceutical composition of claim 17, wherein -Q⁺- is represented by the structural formula: 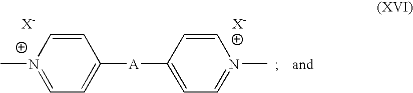
-Q is represented by the structural formula: 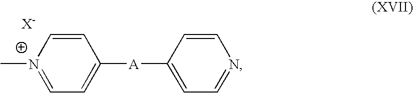
wherein A is a bond or a substituted or unsubstituted lower alkylene or lower alkylene glycol group.

33. The pharmaceutical composition of claim 32, wherein A is an unsubstituted straight chained lower alkylene group.

34. The pharmaceutical composition of claim 33, wherein R₁ is a substituted or unsubstituted straight chained lower alkylene group or polyalkylene glycol optionally substituted with one or more —OH groups and R₁′ is a substituted or unsubstituted straight chained lower alkyl group or polyalkylene glycol, wherein the lower alkyl group or the polyalkylene glycol of R₁′ is optionally substituted with one or more —OH, leaving groups or oxiranyl groups.

35. The pharmaceutical composition of claim 34, wherein R₁ is an unsubstituted straight chained lower alkylene group and R₁′ is an alkyl group substituted with a leaving group, wherein the leaving group is bromine, chlorine, or iodine.

36. The pharmaceutical composition of claim 34, wherein R₁ is an unsubstituted polyalkylene glycol or —CH₂CHOH(CH₂)ₙCHOHCH₂— wherein n is an integer from 0 to 8 and R₁′ is a polyalkylene glycol group substituted with an oxiranyl group or an alkyl group substituted with an oxiranyl group and a hydroxyl group.

37. The pharmaceutical composition of claim 33, wherein -Q⁺-R— is represented by the structural formula:

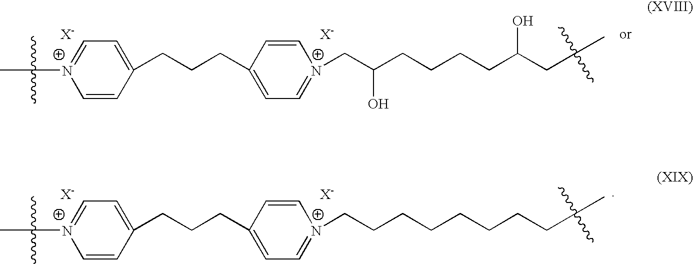

38. The pharmaceutical composition of claim 17, wherein each -Q⁺- is represented by Structural Formula (III) or each -Q⁺- is represented by a structural formula independently selected from Structural Formula (II) and Structural Formula (III) and -Q is represented by Structural Formula (VII) or (VIII).

39. The pharmaceutical composition of claim 17, wherein -Q⁺- is represented by Structural Formula (VI) and -Q is represented by Structural Formula (XI).

40. A method of treating a viral, parasitic or microbial infection in a mammal comprising the step of administering to said mammal an effective amount of a compound comprising molecules represented by Structural Formula (I): 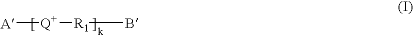
or a pharmaceutically acceptable salt thereof, wherein:
-A′ is —R₁′ or —R₁-Q;
—B′ is -Q⁺-R₁′ or -Q;
each —R₁— is independently a linker and is independently selected;
—R₁′ is a substituted or unsubstituted hydrocarbyl group optionally interrupted with one or more heteroatoms; and
each Q⁺ is independently represented by a structural formula selected from: 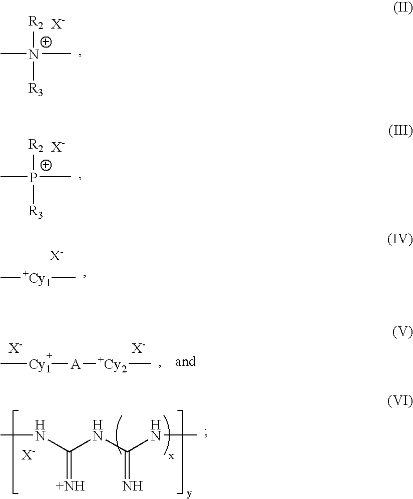
-Q is represented by a structural formula selected from: 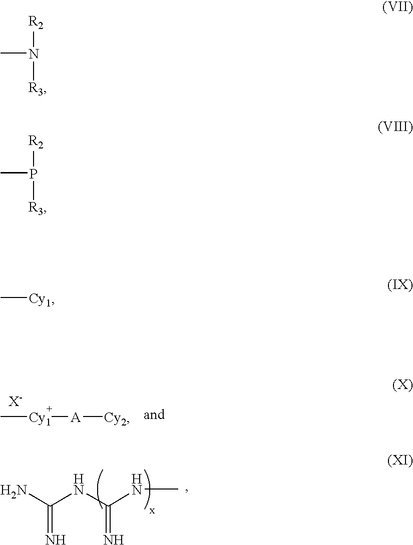
wherein tertiary phosphorus atoms of Structural Formula (VIII), tertiary nitrogen atoms of Structural Formulas (VII), (IX) and (X) and primary nitrogen atoms of Structural Formula (XI) are optionally protonated or alkylated;
each Cy₁⁺ and Cy₂⁺ is independently a quaternary nitrogen-containing monocyclic heteroaromatic ring, a protonated tertiary nitrogen-containing non-aromatic heterocyclic ring or a quaternary nitrogen-containing non-aromatic ring;
each Cy₁ and Cy₂ is independently a nitrogen-containing non-aromatic heterocyclic ring or a nitrogen-containing heteroaromatic ring;
A is a covalent bond, or a substituted or unsubstituted lower alkylene group;
R₂ and R₃ are independently —H or a substituted or unsubstituted aliphatic or aromatic group;
each X⁻, separately or taken together with other X⁻s, is a pharmaceutically acceptable anion;
k is an integer from 1 to 49;
x is an integer from 0-4; and
y is an integer from 1-5.

41. The method of claim 40, wherein k is an integer from 1-15.

42. The method of claim 41, wherein the microbial infection is a bacterial infection.

43. The method of claim 41, wherein the infection is a viral infection.

44. The method of claim 41, wherein the microbial infection is a protozoal infection.

45. The method of claim 41, wherein the microbial infection is a fungal infection.

46. The method of claim 41, wherein the polymer is administered orally, buccally, ophthalmically, topically or by pulmonary means.

47. The method of claim 41, wherein R₁ is a substituted or unsubstituted alkylene group and R₁′ is a substituted or unsubstituted alkyl group.

48. The method of claim 41, wherein each R₂ and R₃ are each independently an alkyl group or a hydroxyalkyl group.

49. The method of claim 48, wherein each -Q⁺- is represented by Structural Formula (II) and each -Q is represented by Structural Formula (VII).

50. The method of claim 49, wherein R₁ is a substituted or unsubstituted straight chained lower alkylene group or polyalkylene glycol, wherein the lower alkylene group or the polyalkylene glycol of R₁ is optionally substituted with one or more —OH groups and R₁′ is a substituted or unsubstituted straight chained lower alkyl group or polyalkylene glycol, wherein the lower alkyl group or the polyalkylene glycol of R₁′ is optionally substituted with one or more —OH, leaving groups or oxiranyl groups.

51. The method of claim 41, wherein -Q⁺- is represented by the structural formula: 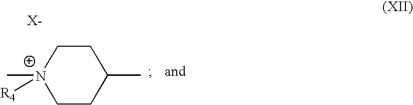
-Q is represented by the structural formula: 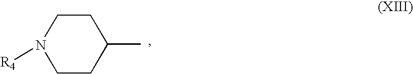
wherein R₄ is —H or a substituted or unsubstituted lower alkyl group.

52. The method of claim 41, wherein -Q⁺- is represented by the structural formula: 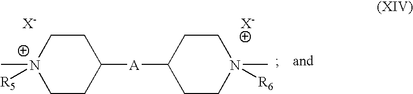
-Q is represented by the structural formula: 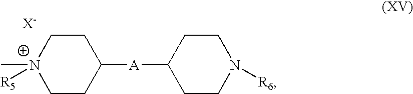
wherein A is a bond or substituted or unsubstituted lower alkylene group, and wherein R₅ and R₆ are each independently —H or a substituted or unsubstituted lower alkyl group.

53. The method of claim 52, wherein R₅ and R₆ are each an alkyl group or a hydroxyalkyl group.

54. The method of claim 53, wherein A is an unsubstituted straight chained lower alkylene group.

55. The method of claim 54, wherein R₁ is a substituted or unsubstituted straight chained lower alkylene group or polyalkylene glycol optionally substituted with one or more —OH groups and R₁′ is a substituted or unsubstituted straight chained lower alkyl group or polyalkylene glycol, wherein the lower alkyl group or the polyalkylene glycol of R₁′ is optionally substituted with one or more —OH, leaving groups or oxiranyl groups.

56. The method of claim 41, wherein -Q⁺- is represented by the structural formula: 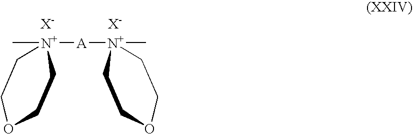
and -Q is represented by the structural formula:

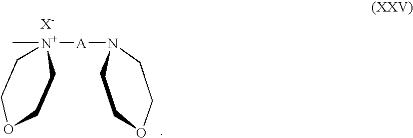

57. The method of claim 41, wherein -Q⁺- is represented by the structural formula: 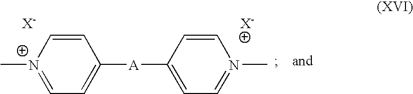
-Q is represented by the structural formula: 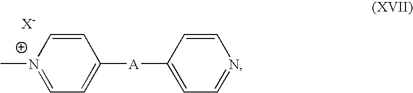
wherein A is a bond or a substituted or unsubstituted lower alkylene or lower alkylene glycol group.

58. The method of claim 57, wherein A is an unsubstituted straight chained lower alkylene group.

59. The method of claim 58, wherein R₁ is a substituted or unsubstituted straight chained lower alkylene group or polyalkylene glycol optionally substituted with one or more —OH groups and R₁′ is a substituted or unsubstituted straight chained lower alkyl group or polyalkylene glycol, wherein the lower alkyl group or the polyalkylene glycol of R₁′ is optionally substituted with one or more —OH, leaving groups or oxiranyl groups.

60. The method of claim 59, wherein -Q⁺-R₁— is represented by the structural formula:

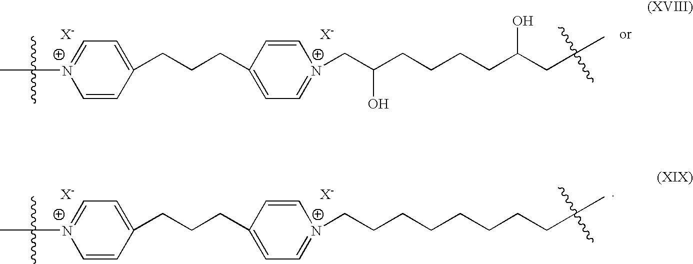

61. The method of claim 41, wherein each -Q⁺- is represented by Structural Formula (III) or each -Q⁺- is represented by a structural formula independently selected from Structural Formula (II) and Structural Formula (III) and -Q is represented by Structural Formula (VII) or (VIII).

62. The method of claim 41, wherein -Q⁺- is represented by Structural Formula (VI) and -Q is represented by Structural Formula (XI).

63. A method of inhibiting the growth of a virus, parasite or microorganism on a surface or the colonization of a surface by a virus, parasite or microorganism comprising the step of contacting said surface with an effective amount of a compound comprising molecules represented by Structural Formula (I): 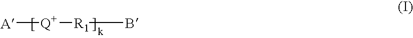
or a salt thereof, wherein:
-A′ is —R₁′ or —R₁-Q;
—B′ is -Q⁺-R′ or -Q;
each —R₁— is independently a substituted hydrocarbylene;
—R₁′ is a substituted or unsubstituted hydrocarbyl group optionally interrupted with one or more heteroatoms; and
each Q⁺ is independently represented by a structural formula selected from: 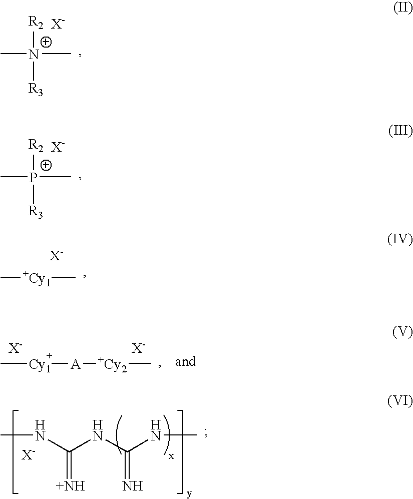
-Q is represented by a structural formula selected from: 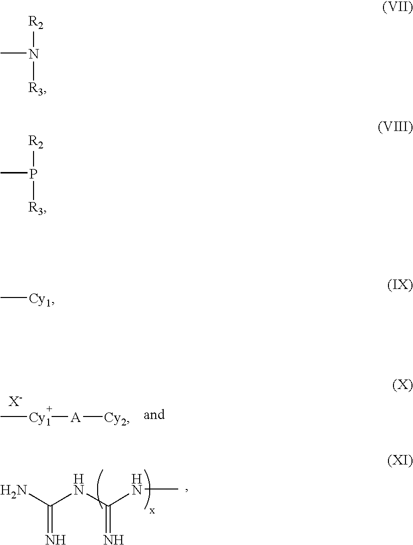
wherein tertiary phosphorus atoms of Structural Formula (VIII), tertiary nitrogen atoms of Structural Formulas (VII), (IX) and (X) and primary nitrogen atoms of Structural Formula (XI) are optionally protonated or alkylated;
each Cy₁⁺ and Cy₂⁺ is independently a quaternary nitrogen-containing monocyclic heteroaromatic ring, a protonated tertiary nitrogen-containing non-aromatic heterocyclic ring or a quaternary nitrogen-containing non-aromatic ring;
each Cy₁ and Cy₂ is independently a nitrogen-containing non-aromatic heterocyclic ring or a nitrogen-containing heteroaromatic ring;
A is a covalent bond, or a substituted or unsubstituted lower alkylene group;
R₂ and R₃ are independently —H or a substituted or unsubstituted aliphatic or aromatic group;
each X⁻, separately or taken together with other X⁻s, is an anion;
k is an integer from 1 to 25;
x is an integer from 0-4; and
y is an integer from 1-5.

64. The method of claim 63, wherein k is an integer from 1-15.

65. The method of claim 64, wherein the surface is in contact with a liquid.

66. The method of claim 64, wherein the surface is in a health-related environment.

67. The method of claim 66, wherein the surface is of a device used in invasive surgical, therapeutic or diagnostic procedures.

68. The method of claim 67, wherein the device is an implantable medical device.

69. The method of claim 64, wherein the surface is a biological barrier for an infectious organism.

70. The method of claim 64, wherein the microorganism is a bacterium.

71. The method of claim 70, wherein the bacterium is a Staphylococcus, Listeria, Bacillus or Salmonella species or E. coli.

72. The method of claim 64, wherein the growth of a virus is inhibited.

73. The method of claim 64, wherein the microorganism is a protist.

74. The method of claim 64, wherein the microorganism is a fungus.

75. The method of claim 64, wherein each R₂ and R₃ are each independently an alkyl group or a hydroxyalkyl group.

76. The method of claim 75, wherein each -Q⁺- is represented by Structural Formula (II) and each -Q is represented by Structural Formula (VII).

77. The method of claim 64, wherein -Q⁺- is represented by the structural formula: 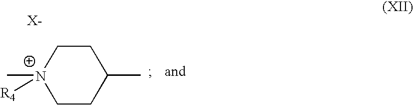
-Q is represented by the structural formula: 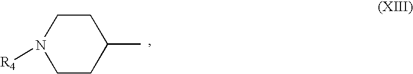
wherein R₄ is —H or a substituted or unsubstituted lower alkyl group.

78. The method of claim 64, wherein -Q⁺- is represented by the structural formula: 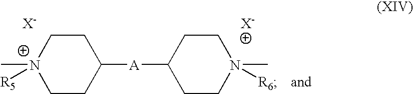
-Q is represented by the structural formula: 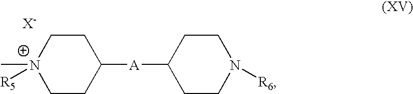
wherein A is a bond or substituted or unsubstituted lower alkylene group, and wherein R₅ and R₆ are each independently —H or a substituted or unsubstituted lower alkyl group.

79. The method of claim 78, wherein R₅ and R₆ are each an alkyl group or a hydroxyalkyl group.

80. The method of claim 79, wherein A is an unsubstituted straight chained lower alkylene group.

81. The method of claim 80, wherein R₁ is a substituted straight chained lower alkylene group or polyalkylene glycol substituted with one or more —OH groups and R₁′ is a substituted or unsubstituted straight chained lower alkyl group or polyalkylene glycol, wherein the lower alkyl group or the polyalkylene glycol of R₁′ is optionally substituted with one or more —OH, leaving groups or oxiranyl groups.

82. The method of claim 64, wherein -Q⁺- is represented by the structural formula: 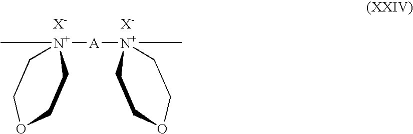
and -Q is represented by the structural formula:

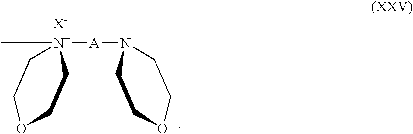

83. The method of claim 64, wherein -Q⁺- is represented by the structural formula: 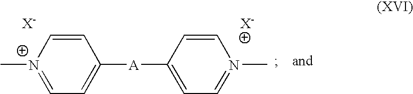
-Q is represented by the structural formula: 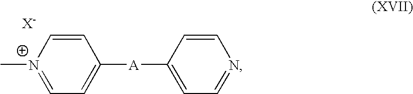
wherein A is a bond or a substituted or unsubstituted lower alkylene or lower alkylene glycol group.

84. The method of claim 83, wherein A is an unsubstituted straight chained lower alkylene group.

85. The method of claim 84, wherein R₁ is a substituted straight chained lower alkylene group or polyalkylene glycol substituted with one or more OH groups and R₁′ is a substituted or unsubstituted straight chained lower alkyl group or polyalkylene glycol, wherein the lower alkyl group or the polyalkylene glycol of R₁′ is optionally substituted with one or more —OH, leaving groups or oxiranyl groups.

86. The method of claim 85, wherein -Q⁺-R₁— is represented by the structural formula:

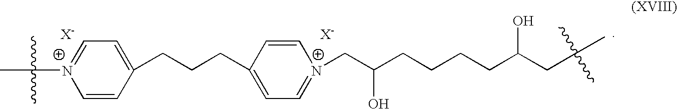

87. The method of claim 64, wherein each -Q⁺- is represented by Structural Formula (III) or each -Q⁺- is represented by a structural formula independently selected from Structural Formula (II) and Structural Formula (III) and -Q is represented by Structural Formula (VII) or (VIII).

88. The method of claim 64, wherein -Q⁺- is represented by Structural Formula (VI) and -Q is represented by Structural Formula (XI).

89. A method of treating mucositis in a mammal comprising the step of administering to said mammal an effective amount of a compound comprising molecules represented by Structural Formula (I): 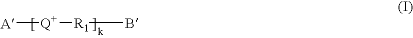
or a pharmaceutically acceptable salts thereof, wherein:
-A′ is —R₁′ or —R₁-Q;
—B′ is -Q⁺-R′ or -Q;
each —R₁— is independently a linker;
—R₁′ is a substituted or unsubstituted hydrocarbyl group optionally interrupted with one or more heteroatoms; and
each Q⁺ is independently represented by a structural formula selected from: 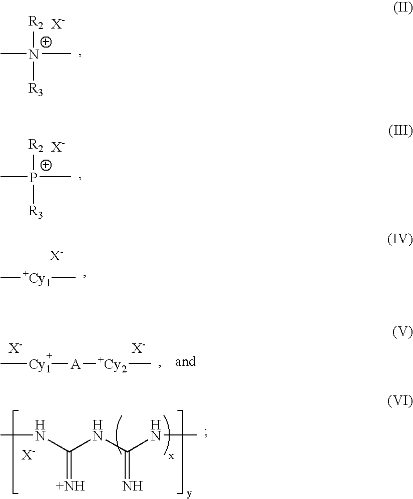
-Q is represented by a structural formula selected from: 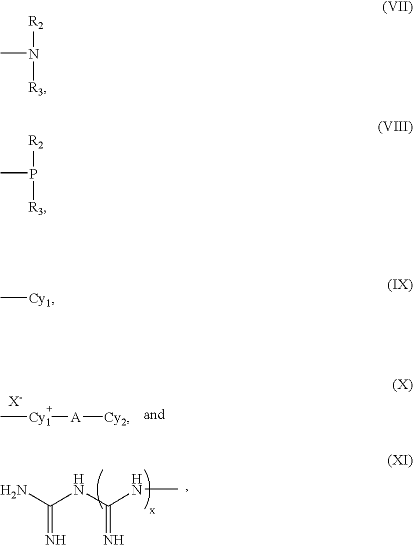
wherein tertiary phosphorus atoms of Structural Formula (VIII), tertiary nitrogen atoms of Structural Formulas (VII), (IX) and (X) and primary nitrogen atoms of Structural Formula (XI) are optionally protonated or alkylated;
each Cy₁⁺ and Cy₂⁺ is independently a quaternary nitrogen-containing monocyclic heteroaromatic ring, a protonated tertiary nitrogen-containing non-aromatic heterocyclic ring or a quaternary nitrogen-containing non-aromatic ring;
each Cy₁ and Cy₂ is independently a nitrogen-containing non-aromatic heterocyclic ring or a nitrogen-containing heteroaromatic ring;
A is a covalent bond, or a substituted or unsubstituted lower alkylene group;
R₂ and R₃ are independently —H or a substituted or unsubstituted aliphatic or aromatic group;
each X⁻, separately or taken together with other X⁻s, is a pharmaceutically acceptable anion;
k is an integer from 1 to 25;
x is an integer from 0-4; and
y is an integer from 1-5.

90. The method of claim 89, wherein k is an integer from 1-15.

91. The method of claim 90, wherein the polymer is administered therapeutically.

92. The method of claim 90, wherein the polymer is administered prophylactically.

93. The method of claim 90, wherein said mucositis is oral mucositis.

94. The method of claim 93, wherein said oral mucositis is a side effect of anti-cancer therapy.

95. The method of claim 94, wherein said anti-cancer therapy is chemotherapy or radiation therapy.

96. The method of claim 93, wherein said oral mucositis is a side effect of bone marrow transplantation or stem cell transplantation or ablation.

97. The method of claim 90, wherein R₁ is a substituted or unsubstituted alkylene group and R₁′ is a substituted or unsubstituted alkyl group.

98. The method of claim 90, wherein each R₂ and R₃ are each independently an alkyl group or a hydroxyalkyl group.

99. The method of claim 98, wherein each -Q⁺- is represented by Structural Formula (II) and each -Q is represented by Structural Formula (VII).

100. The method of claim 99, wherein R₁ is an unsubstituted straight chained lower alkylene group and R₁′ is an alkyl group substituted with a leaving group, wherein the leaving group is bromine, chlorine, or iodine.

101. The method of claim 90, wherein -Q⁺- is represented by the structural formula: 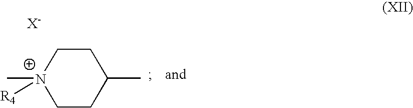
-Q is represented by the structural formula: 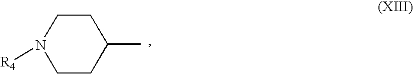
wherein R₄ is —H or a substituted or unsubstituted lower alkyl group.

102. The method of claim 90, wherein -Q⁺- is represented by the structural formula: 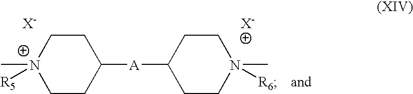
-Q is represented by the structural formula: 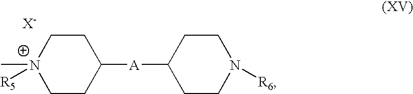
wherein A is a bond or substituted or unsubstituted lower alkylene group, and wherein R₅ and R₆ are each independently —H or a substituted or unsubstituted lower alkyl group.

103. The method of claim 102, wherein R₅ and R₆ are each an alkyl group or a hydroxyalkyl group.

104. The method of claim 103, wherein A is an unsubstituted straight chained lower alkylene group.

105. The method of claim 104, wherein R₁ is a substituted or unsubstituted straight chained lower alkylene group or polyalkylene glycol optionally substituted with one or more —OH groups and R₁′ is a substituted or unsubstituted straight chained lower alkyl group or polyalkylene glycol, wherein the lower alkyl group or the polyalkylene glycol of R₁′ is optionally substituted with one or more —OH, leaving groups or oxiranyl groups.

106. The method of claim 90, wherein -Q⁺- is represented by the structural formula: 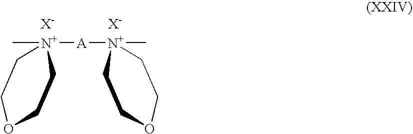
and -Q is represented by the structural formula:

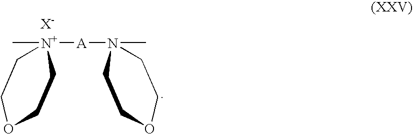

107. The method of claim 90, wherein -Q⁺- is represented by the structural formula: 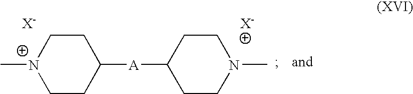
-Q is represented by the structural formula: 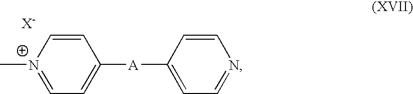
wherein A is a bond or a substituted or unsubstituted lower alkylene or lower alkylene glycol group.

108. The method of claim 107, wherein A is an unsubstituted straight chained lower alkylene group.

109. The method of claim 108, wherein R₁ is a substituted or unsubstituted straight chained lower alkylene group or polyalkylene glycol optionally substituted with one or more —OH groups and R₁′ is a substituted or unsubstituted straight chained lower alkyl group or polyalkylene glycol, wherein the lower alkyl group or the polyalkylene glycol of R₁′ is optionally substituted with one or more —OH, leaving groups or oxiranyl groups.

110. The method of claim 109, wherein -Q⁺-R₁— is represented by the structural formula:

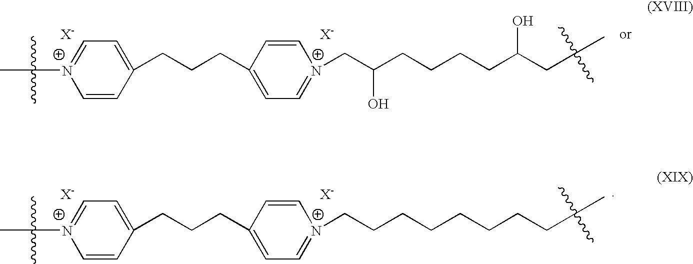

111. The method of claim 90, wherein each -Q⁺- is represented by Structural Formula (III) or each -Q⁺- is represented by a structural formula independently selected from Structural Formula (II) and Structural Formula (III) and -Q is represented by Structural Formula (VII) or (VIII).

112. The method of claim 90, wherein -Q⁺- is represented by Structural Formula (VI) and -Q is represented by Structural Formula (XI).

113. A method of preventing or treating infection or colonization in a cystic fibrosis patient comprising the step of administering to said mammal an effective amount of a compound comprising molecules represented by Structural Formula (I): 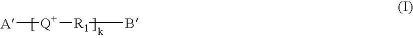
or a pharmaceutically acceptable salts thereof, wherein:
-A′ is —R₁′ or —R₁-Q;
—B′ is -Q⁺-R₁′ or -Q;
each —R₁— is independently a linker;
—R₁′ is a substituted or unsubstituted hydrocarbyl group optionally interrupted with one or more heteroatoms; and
each Q⁺ is independently represented by a structural formula selected from: 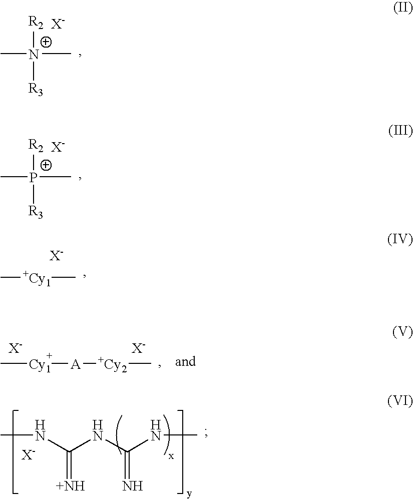
-Q is represented by a structural formula selected from: 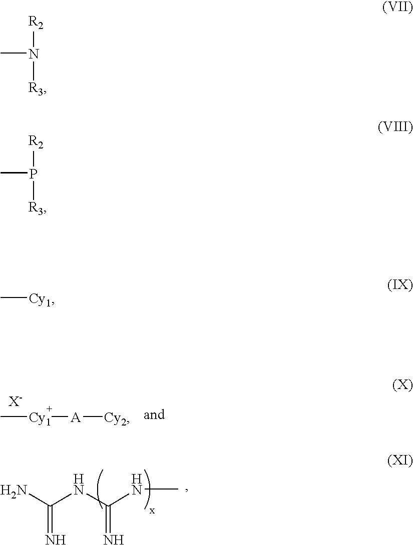
wherein tertiary phosphorus atoms of Structural Formula (VIII), tertiary nitrogen atoms of Structural Formulas (VII), (IX) and (X) and primary nitrogen atoms of Structural Formula (XI) are optionally protonated or alkylated;
each Cy₁⁺ and Cy₂⁺ is independently a quaternary nitrogen-containing monocyclic heteroaromatic ring, a protonated tertiary nitrogen-containing non-aromatic heterocyclic ring or a quaternary nitrogen-containing non-aromatic ring;
each Cy₁ and Cy₂ is independently a nitrogen-containing non-aromatic heterocyclic ring or a nitrogen-containing heteroaromatic ring;
A is a covalent bond, or a substituted or unsubstituted lower alkylene group;
R₂ and R₃ are independently —H or a substituted or unsubstituted aliphatic or aromatic group;
each X⁻, separately or taken together with other X⁻s, is a pharmaceutically acceptable anion;
k is an integer from 1 to 25;
x is an integer from 0-4; and
y is an integer from 1-5.

114. The method of claim 113, wherein k is an integer from 1-15.

115. The method of claim 114, wherein the mammal is suffering from a pulmonary infection.

116. The method of claim 115, wherein the polymer is administered as an aerosol.

117. The method of claim 116, wherein the molecular weight of the polymer is 1000 to 3000 Daltons.

118. The method of claim 115, wherein the pulmonary infection or colonization is caused by a microbe selected from the group consisting of Pseudomonas, Staphylococcus, Haemophilus, Burkholderia, Aspergillus, Candida, Mycobacteria, Mycoplasma, Stenotrophomonas, Escherichia, Achromobacter, Ralstonia, Acinetobacter, Streptococcus, Flavobacterium or Klebsiella species, and combinations thereof.

119. The method of claim 118, wherein the microbe is selected from the group consisting of Pseudomonas aeruginosa, Staphylococcus aureus, Haemophilus influenzae, Burkholderia cepacia, Aspergillus fumigatus, Candida albicans, Mycoplasma pneumoniae, Stenotrophomonas maltophilia, Escherichia coli, Klebsiella pneumoniae, Ralstonia mannitolilytica, Ralstonia pickettii, Streptococcus pneumoniae, Flavobacterium indologenes, Burkholderia gladioli, Acinetobacter baumannii, Achromobacter xylosoxidans and combinations thereof.

120. The method of claim 116, wherein R₁ is a substituted or unsubstituted alkylene group and R₁′ is a substituted or unsubstituted alkyl group.

121. The method of claim 116, wherein each R₂ and R₃ are each independently an alkyl group or a hydroxyalkyl group.

122. The method of claim 121, wherein each -Q⁺- is represented by Structural Formula (II) and each -Q is represented by Structural Formula (VII).

123. The method of claim 122, wherein R₁ is an unsubstituted straight chained lower alkylene group and R₁′ is an alkyl group substituted with a leaving group, wherein the leaving group is bromine, chlorine, or iodine.

124. The method of claim 116, wherein -Q⁺- is represented by the structural formula: 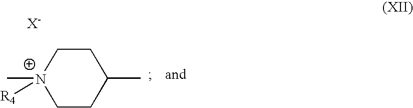
-Q is represented by the structural formula: 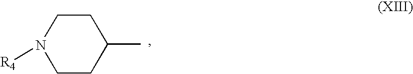
wherein R₄ is —H or a substituted or unsubstituted lower alkyl group.

125. The method of claim 116, wherein -Q⁺- is represented by the structural formula: 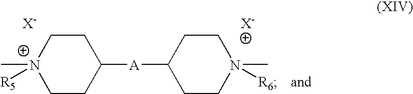
-Q is represented by the structural formula: 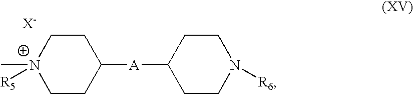
wherein A is a bond or substituted or unsubstituted lower alkylene group, and wherein R₅ and R₆ are each independently —H or a substituted or unsubstituted lower alkyl group.

126. The method of claim 125, wherein R₅ and R₆ are each an alkyl group or a hydroxyalkyl group.

127. The method of claim 126, wherein A is an unsubstituted straight chained lower alkylene group.

128. The method of claim 127, wherein R₁ is a substituted or unsubstituted straight chained lower alkylene group or polyalkylene glycol optionally substituted with one or more —OH groups and R₁′ is a substituted or unsubstituted straight chained lower alkyl group or polyalkylene glycol, wherein the lower alkyl group or the polyalkylene glycol of R₁′ is optionally substituted with one or more —OH, leaving groups or oxiranyl groups.

129. The method of claim 116, wherein -Q⁺- is represented by the structural formula: 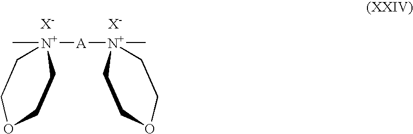
and -Q is represented by the structural formula:

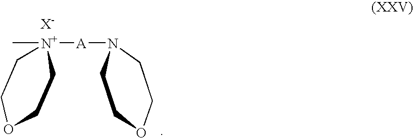

130. The method of claim 116, wherein -Q⁺- is represented by the structural formula: 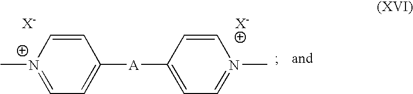
-Q is represented by the structural formula: 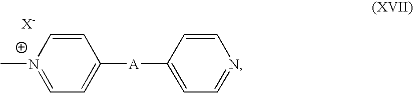
wherein A is a bond or a substituted or unsubstituted lower alkylene or lower alkylene glycol group.

131. The method of claim 130, wherein A is an unsubstituted straight chained lower alkylene group.

132. The method of claim 131, wherein R₁ is a substituted or unsubstituted straight chained lower alkylene group or polyalkylene glycol optionally substituted with one or more —OH groups and R₁′ is a substituted or unsubstituted straight chained lower alkyl group or polyalkylene glycol, wherein the lower alkyl group or the polyalkylene glycol of R₁′ is optionally substituted with one or more —OH, leaving groups or oxiranyl groups.

133. The method of claim 132, wherein -Q⁺-R₁— is represented by the structural formula:

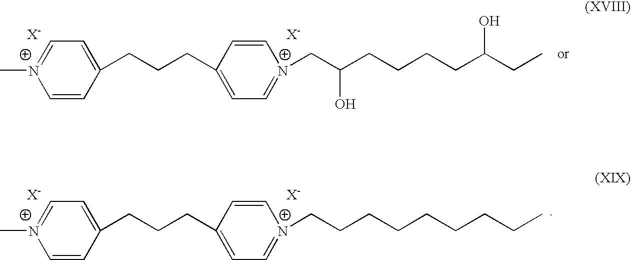

134. The method of claim 116, wherein each -Q ⁺- is represented by Structural Formula (III) or each -Q⁺- is represented by a structural formula independently selected from Structural Formula (II) and Structural Formula (III) and -Q is represented by Structural Formula (VII) or (VIII).

135. The method of claim 116, wherein -Q⁺- is represented by Structural Formula (VI) and -Q is represented by Structural Formula (XI).

136. A polymer comprised of repeat units represented by Structural Formula (XX): 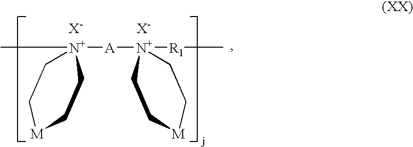
wherein:
each —R₁— is independently a linker;
A is a covalent bond, or a substituted or unsubstituted lower alkylene or arylene group;
M is (CH₂)ₜ, O, N, S, SO or SO₂;
each X⁻, separately or taken together with other X⁻s, is an anion;
j is a positive integer; and
t is 0 or 1.

137. The polymer of claim 136, wherein j is from 50 to 500.

138. The polymer of claim 137, wherein R₁ is a substituted or unsubstituted alkylene group.

139. The polymer of claim 138, wherein A is a bond or a substituted or unsubstituted alkylene group.

140. The polymer of claim 139, wherein M is O.

141. A pharmaceutical composition comprising a carrier or diluent and a polymer comprised of repeat units represented by the structural formula: 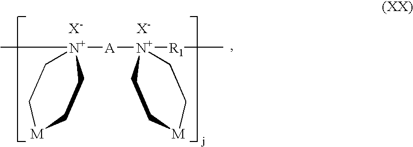
wherein:
each —R₁— is independently a linker;
A is a covalent bond, or a substituted or unsubstituted lower alkylene or arylene group;
M is (CH₂)ₜ, O, N, S, SO or SO₂;
each X⁻, separately or taken together with other X⁻s, is an anion;
j is a positive integer; and
t is 0 or 1.

142. A method of treating a viral, parasitic or microbial infection in a mammal comprising the step of administering to said mammal an effective amount of a polymer comprised of repeat units represented by the structural formula: 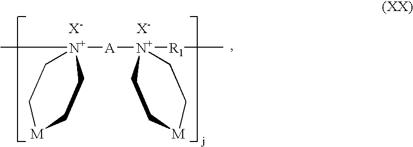
wherein:
each —R₁— is independently a linker;
A is a covalent bond, or a substituted or unsubstituted lower alkylene or arylene group;
M is (CH₂)ₜ, O, N, S, SO or SO₂;
each X⁻, separately or taken together with other X⁻s, is an anion;
j is a positive integer; and
t is 0 or 1.

143. A method of inhibiting the growth of a virus, parasite or microorganism on a surface comprising the step of contacting said surface with an effective amount of a polymer comprised of repeat units represented by the structural formula: 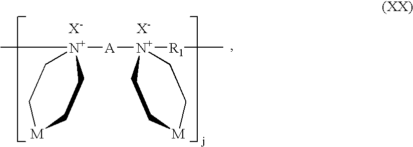
wherein:
each —R₁— is independently a linker;
A is a covalent bond, or a substituted or unsubstituted lower alkylene or arylene group;
M is (CH₂)ₜ, O, N, S, SO or SO₂;
each X⁻, separately or taken together with other X⁻s, is an anion;
j is a positive integer; and
t is 0 or 1.

144. A method of treating mucositis in a mammal comprising the step of administering to said mammal an effective amount of a polymer comprised of repeat units represented by the structural formula: 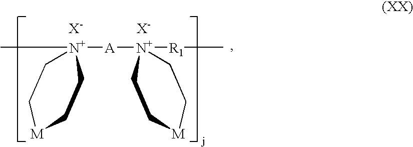
wherein:
each —R₁— is independently a linker;
A is a covalent bond, or a substituted or unsubstituted lower alkylene or arylene group;
M is (CH₂)ₜ, O, N, S, SO or SO₂;
each X⁻, separately or taken together with other X⁻s, is an anion;
j is a positive integer; and
t is 0 or 1.

145. A method of preventing or treating infection or colonization in a cystic fibrosis patient comprising the step of administering to said mammal an effective amount of a polymer comprised of repeat units represented by the structural formula: 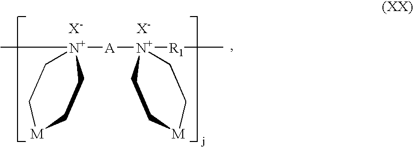
wherein:
each —R₁— is independently a linker;
A is a covalent bond, or a substituted or unsubstituted lower alkylene or arylene group;
M is (CH₂), O, N, S, SO or SO₂;
each X⁻, separately or taken together with other X⁻s, is an anion;
j is a positive integer; and
t is 0 or 1.

146. A polymer comprised of repeat units represented by Structural Formula (XXI): 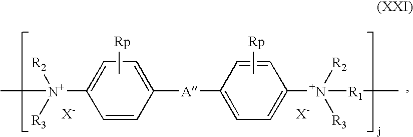
wherein:
each —R₁— is independently a linker;
R₂ and R₃ are independently —H or a substituted or unsubstituted aliphatic or aromatic group;
Rₚ is independently —H, a halogen, or a substituted or unsubstituted alkyl or alkyloxy group;
A″ is a covalent bond, or a substituted or unsubstituted lower alkylene or alkenylene group of two or more carbons; and
each X⁻, separately or taken together with other X⁻s, is an anion; and
j is a positive integer.

147. The polymer of claim 146, wherein j is from 50 to 500.

148. The polymer of claim 146, wherein j is from 1 to 49.

149. The polymer of claim 148, wherein j is from 1 to 15.

150. The polymer of claim 148, further comprising two capping groups represented by structural formula: 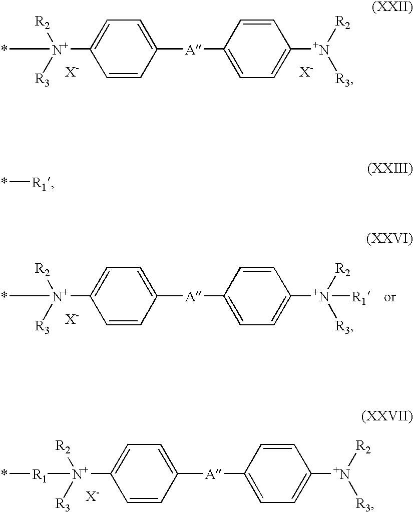
wherein R₁′ is a substituted or unsubstituted alkyl or aryl group and * represents where the capping group is attached to the polymer.

151. The polymer of claim 146, wherein R₁ is a substituted or unsubstituted alkylene group.

152. The polymer of claim 151, wherein A″ is a bond or a substituted or unsubstituted alkylene group.

153. The polymer of claim 152, wherein R₂ and R₃ are each independently an alkyl group or a hydroxyalkyl group.

154. A pharmaceutical composition comprising a carrier or diluent and a polymer comprised of repeat units represented by the structural formula: 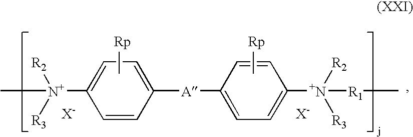
wherein:
each —R₁— is independently a linker;
R₂ and R₃ are independently —H or a substituted or unsubstituted aliphatic or aromatic group;
Rₚ is independently —H, a halogen or a substituted or unsubstituted alkyl or alkoxy group;
A″ is a covalent bond, or a substituted or unsubstituted lower alkylene or alkenylene group;
each X⁻, separately or taken together with other X⁻s, is a pharmaceutically acceptable anion; and
j is a positive integer.

155. A method of treating a viral, parasitic or microbial infection in a mammal comprising the step of administering to said mammal an effective amount of a polymer comprised of repeat units represented by the structural formula: 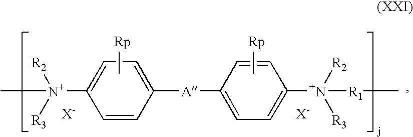
wherein:
each —R₁— is independently a linker;
R₂ and R₃ are independently —H or a substituted or unsubstituted aliphatic or aromatic group;
Rₚ is independently —H, a halogen or a substituted or unsubstituted alkyl or alkoxy group;
A″ is a covalent bond, or a substituted or unsubstituted lower alkylene or alkenylene group;
each X⁻, separately or taken together with other X⁻s, is a pharmaceutically acceptable anion; and
j is a positive integer.

156. A method of inhibiting the growth of a microorganism, parasite or virus on a surface comprising the step of contacting said surface with an effective amount of a polymer comprised of repeat units represented by the structural formula: 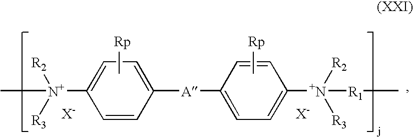
wherein:
each —R₁— is independently a linker;
R₂ and R₃ are independently —H or a substituted or unsubstituted aliphatic or aromatic group;
Rₚ is independently —H, a halogen or a substituted or unsubstituted alkyl or alkoxy group;
A″ is a covalent bond, or a substituted or unsubstituted lower alkylene or alkenylene group;
each X⁻, separately or taken together with other X⁻s, is an anion; and
j is a positive integer.

157. A method of treating mucositis in a mammal comprising the step of administering to said mammal an effective amount of a polymer comprised of repeat units represented by the structural formula: 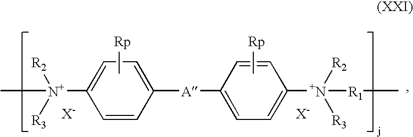
wherein:
each —R₁— is independently a linker;
R₂ and R₃ are independently —H or a substituted or unsubstituted aliphatic or aromatic group;
Rₚ is independently —H, a halogen or a substituted or unsubstituted alkyl or alkoxy group;
A″ is a covalent bond, or a substituted or unsubstituted lower alkylene or alkenylene group;
each X⁻, separately or taken together with other X⁻s, is a pharmaceutically acceptable anion; and
j is a positive integer.

158. A method of preventing or treating infection or colonization in a cystic fibrosis patient comprising the step of administering to said mammal an effective amount of a polymer comprised of repeat units represented by the structural formula: 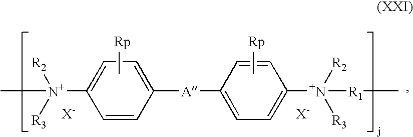
wherein:
each —R₁— is independently a linker;
R₂ and R₃ are independently —H or a substituted or unsubstituted aliphatic or aromatic group;
Rₚ is independently —H, a halogen or a substituted or unsubstituted alkyl or alkoxy group;
A″ is a covalent bond, or a substituted or unsubstituted lower alkylene or alkenylene group;
each X⁻, separately or taken together with other X⁻s, is a pharmaceutically acceptable anion; and
j is a positive integer.
